(19) United States
(12) Patent Application Publication
(10) Pub. No.: US 2008/0255137 A1
(43) Pub. Date: Oct. 16, 2008

(54) N-Biaryl and N-Arylheteroaryl 2-Substituted Piperazine Derivatives as Modulators of the 5ht2c Receptor Useful for the Treatment of Disorders Related Thereto

(75) Inventors: Brian M Smith, San Diego, CA (US); Vincent J. Santora, Carlsbad, CA (US); Rena Hayashi, San Diego, CA (US); Jason B. Ibarra, Las Vegas, NV (US); Jeffrey A. Schultz, San Diego, CA (US); Scott A. Estrada, La Jolla, CA (US)

Correspondence Address: FISH & RICHARDSON P.C., P.O. BOX 1022, MINNEAPOLIS, MN, 55440-1022, US

(73) Assignee: Arena Pharmaceuticals, Inc., San Diego, CA (US)

(21) Appl. No.: 11/792,742

(22) PCT Filed: Dec. 9, 2005
(86) PCT No.: PCT/US05/44815

§ 371 Date: Mar. 25, 2008

Related U.S. Application Data
(60) Provisional application No. 60/635,794, filed on Dec. 13, 2004.

(51) Int. Cl. A61K 31/496; C07D 409/10; C07D 401/10; C07D 405/10; C07D 241/04; A61P 3/00; A61P 25/00

(52) U.S. Cl. 514/252.13; 544/379; 544/360; 514/253.01; 514/254.1; 544/403; 514/255.03

(57) ABSTRACT

The present invention relates to certain N-biaryl and N-arylheteroaryl 2-substituted piperazine derivatives of Formula (Ia) that are modulators of the 5HT2C receptor. Accordingly, compounds of the present invention are useful for the treatment of 5HT2C receptor associated diseases or disorders, such as, obesity, Alzheimer Disease, erectile dysfunction and related disorders.

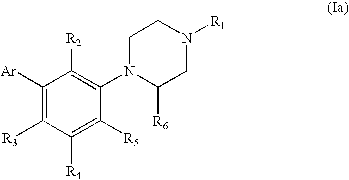

### FIELD OF THE INVENTION

The present invention relates to certain N-biaryl and N-arylheteroaryl 2-substituted piperazine derivatives that are modulators of the 5HT_{2C} receptor. Accordingly, compounds of the present invention are useful for the treatment of 5HT_{2C} receptor associated diseases or disorders, such as, obesity, Alzheimer Disease, erectile dysfunction and related disorders.

### BACKGROUND OF THE INVENTION

Obesity is a life-threatening disorder in which there is an increased risk of morbidity and mortality arising from concomitant diseases such as, but not limited to, type II diabetes, hypertension, stroke, certain forms of cancers and gallbladder disease.

Obesity has become a major healthcare issue in the Western World and increasingly in some third world countries. The increase in the number of obese people is due largely to the increasing preference for high fat content foods but also, and this can be a more important factor, the decrease in activity in most people's lives. In the last 10 years there has been a 30% increase in the incidence of obesity in the USA and that about 30% of the population of the USA is now considered obese. In spite of the growing awareness of the health concerns linked to obesity the percentage of individuals that are overweight or obese continue to increase. In fact, the percentage of children and adolescents who are defined as overweight has more than doubled since the early 1970s and about 13 percent of children and adolescents are now seriously overweight. The most significant concern, from a public health perspective, is that children who are overweight grow up to be overweight or obese adults, and accordingly are at greater risk for major health problems. Therefore, it appears that the number of individuals that are overweight or obese will continue to increase.

Whether someone is classified as overweight or obese is generally determined on the basis of his or her body mass index (BMI) which is calculated by dividing their body weight (kilograms—Kg) by their height squared (meters squared—m²). Thus, the units for BMI are Kg/m². The BMI is more highly correlated with body fat than any other indicator of height and weight. A person is considered overweight when they have a BMI in the range of 25-30 kg/m². Whereas a person with a BMI over 30 kg/m² is classified as obese and obesity is further divided into three classes, Class I (BMI of about 30 to about 34.9 kg/m²), Class II (BMI of about 35 to 39.9 kg/m²) and Class III (about 40 kg/m² or greater); see TABLE 1 below for complete classifications.

TABLE 1
CLASSIFICATION OF WEIGHT BY BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases for an individual there is an increased risk of morbidity and mortality relative to an individual with normal BMI. Accordingly, overweight and obese individuals (BMI of about 25 kg/m² and above) are at increased risk for physical ailments such as, but not limited to, high blood pressure, cardiovascular disease (particularly hypertension), high blood cholesterol, dyslipidemia, type II (non-insulin dependent) diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), diseases of reproduction (such as sexual dysfunction, both male and female, including male erectile dysfunction), bladder control problems (such as stress incontinence), uric acid nephrolithiasis, psychological disorders (such as depression, eating disorders, distorted body image, and low self esteem). Research has shown that even a modest reduction in body weight can correspond to a significant reduction in the risk of developing other ailments, such as, but not limited to, coronary heart disease.

As mentioned above, obesity increases the risk of developing cardiovascular diseases. Coronary insufficiency, atheromatous disease, and cardiac insufficiency are at the forefront of the cardiovascular complications induced by obesity. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions based on the prevention of obesity [Perry, I. J., et al. BMJ 310, 560-564 (1995)]. It is estimated that if the entire population had an ideal weight, the risk of coronary insufficiency would decrease by 25% and the risk of cardiac insufficiency and of cerebral vascular accidents by 35%.

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina and increases the risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

The first line of treatment for individuals that are overweight or obese is to offer diet and life style advice, such as, reducing the fat content of their diet and increasing their physical activity. However many patients find these difficult to maintain and need additional help from drug therapy to sustain results from these efforts.

Most currently marketed products have been unsuccessful as treatments for obesity owing to a lack of efficacy or unacceptable side-effect profiles. The most successful drug so far was the indirectly acting 5-hydroxytryptamine (5-HT) agonist d-fenfluramine (Redux™) but reports of cardiac valve defects in up to one third of the patient population led to its withdrawal by the FDA in 1998.

In addition, two drugs have recently been launched in the USA and Europe: Orlistat (Xenical™), a drug that prevents absorption of fat by the inhibition of pancreatic lipase, and Sibutramine (Reductil™), a 5-HT/noradrenaline re-uptake inhibitor. However, side effects associated with these products may limit their long-term utility. Treatment with Xenical™ is reported to induce gastrointestinal distress in some patients, while Sibutramine has been associated with raised blood pressure in some patients.

Serotonin (5-HT) neurotransmission plays an important role in numerous physiological processes both in health and in psychiatric disorders. 5-HT has been implicated in the regulation of feeding behavior for some time. 5-HT works by inducing a feeling of fullness or satiety so eating stops earlier and fewer calories are consumed. It has been shown that a stimulatory action of 5-HT on the 5HT_{2C} receptor plays an important role in the control of eating and in the anti-obesity effect of d-fenfluramine. As the 5HT_{2C} receptor is expressed in high density in the brain (notably in the limbic structures, extrapyramidal pathways, thalamus and hypothalamus i.e. PVN and DMH, and predominantly in the choroid plexus) and is expressed in low density or is absent in peripheral tissues, a selective 5HT_{2C} receptor agonist can be an effective and safe anti-obesity agent. Also, 5HT_{2C} knockout mice are overweight with cognitive impairment and susceptibility to seizure thus establishing the clear use for a 5HT_{2C} receptor agonist in 5HT_{2C} receptor associated diseases or disorders.

The 5HT_{2C} receptor plays a role in obsessive compulsive disorder, some forms of depression, and epilepsy. Accordingly, 5HT_{2C} receptor agonists can have anti-panic properties, and properties useful for the treatment of sexual dysfunction. In addition, 5HT_{2C} receptor agonists are useful for the treatment of psychiatric symptoms and behaviors in individuals with eating disorders such as, but not limited to, anorexia nervosa and bulimia nervosa. Individuals with anorexia nervosa often demonstrate social isolation. Anorexic individuals often present symptoms of being depressed, anxious, obsession, perfectionistic traits, and rigid cognitive styles as well as sexual disinterest. Other eating disorders include, anorexia nervosa, bulimia nervosa, binge eating disorder (compulsive eating) and ED-NOS (i.e., eating disorders not otherwise specified—an official diagnosis). An individual diagnosed with ED-NOS possess atypical eating disorders including situations in which the individual meets all but a few of the criteria for a particular diagnosis. What the individual is doing with regard to food and weight is neither normal nor healthy.

In addition, the 5HT_{2C} receptor is also involved in other diseases, conditions and disorders; such as Alzheimer Disease (AD). Therapeutic agents currently prescribed for Alzheimer's disease (AD) are cholinomimetic agents that act by inhibiting the enzyme acetylcholinesterase. The resulting effect is increased levels of acetylcholine, which modestly improves neuronal function and cognition in patients with AD. Although, dysfunction of cholinergic brain neurons is an early manifestation of AD, attempts to slow the progression of the disease with these agents have had only modest success, perhaps because the doses that can be administered are limited by peripheral cholinergic side effects, such as tremors, nausea, vomiting, and dry mouth. In addition, as AD progresses, these agents tend to lose their effectiveness due to continued cholinergic neuronal loss.

Therefore, there is a need for agents that have beneficial effects in AD, particularly in alleviating symptoms by improving cognition and slowing or inhibiting disease progression, without the side effects observed with current therapies. Therefore, serotonin 5HT_{2C} receptors, which are exclusively expressed in brain, are attractive targets.

A major feature of AD is the formation of senile plaques made of amyloid deposits in a selected area of the brain. New therapies should focus on prevention of the production of these senile plaques. An amyloid deposit composed mainly of beta-amyloid peptide (Aβ) occupies the plaque center. Aβ is a peptide of 40 to 43 residues derived from a larger amyloid precursor protein, APP [Selkoe D J, et al. Ann Rev Neurosci, 1994, 17:489-517]. APP is a ubiquitous transmembrane glycoprotein that is present at high levels in brain cells. APP also exists as secreted forms. By cleavage in the Aβ region of APP, the long N-terminal fragment (secreted APP, APPs) is secreted into the extracellular space. The rate of Aβ production appears to be inversely coupled to rate APPs secretion. In several cell cultures, APPs secretion was accompanied by reductions in secreted Aβ [Buxbaum J D, et al. Proc Nat Acad Sci, 1993, 90:9195-9198; Gabuzda D, et al. J Neurochem, 1993, 61:2326-2329; Hung A Y, et al. J Biol Chem, 1993, 268:22959-22962; and Wolf B A, et al. J Biol Chem, 1995, 270:4916-4922], suggesting that stimulated secretory processing of APP into secreted APPs is associated with reduced formation of potentially amyloidogenic derivatives, or plaques.

APPs is found in plasma and cerebrospinal fluid [Ghiso J, et al. Biochem Biophys Res Comm, 1989, 163:430-437; and Podlisny M B, et al. Biochem Biophys Res Commun, 1990, 167:1094-1101]. Considering the abundance of both membrane-bound APP and APPs, they are likely to have significant biological functions. Current knowledge about APP functions indicates APP is critically required for the maintenance of neuronal and synaptic structure and function. Membrane-bound APP has been suggested to have a receptor-like structure [Kang J, et al. Nature, 1987, 325:733-736], with the cytoplasmic domain capable of complexing with a GTP-binding protein [Nishimoto I., et al. Nature, 1993, 362:75-79]. Membrane-embedded full-length APP might also have a cell adhesion function [Qiu W., et al. J Neurosci, 1995, 15:2157-2167].

APPs has been shown to be neurotrophic and neuroprotective in vitro [Mattson M P, et al. Neuron, 1993, 10:243-254; and Qiu W., et al. J Neurosci, 1995, 15:2157-2167]. Other proposed functions for APPs include the regulation of blood coagulation [Cole G M, et al. Biochem Biophys Res Commun, 1990, 170:288-295; Smith R P, et al. Science, 1990, 248:1126-1128; and Van Nostrand et al. Science, 1990, 248:745-748], wound-healing [Cunningham J M, et al. Histochemistry, 1991, 95:513-517], extracellular protease activity [Oltersdorf T, et al. Nature (London), 1989, 341:144-147; and Van Nostrand W E, et al. Nature, 1989, 341:546-548], neurite extension [Jin L., et al. J Neurosci, 1994, 14:5461-5470; and Robakis N K, et al. in Molecular Biology of Alzheimer's Disease. (T. Miyatake, D. J. Selkoe and Y. Ihara, ed.), 1990, pp. 179-188, Elsevier Science Publishers B.V., Amsterdam], cell adhesiveness [Schubert D, et al. Neuron, 1989, 3:689-694], cell growth, [Bhasin R., et al. Proc Natl Acad Sci USA, 1991, 88:10307-10311; and Saitoh T., Cell, 1989, 58:615-622], and differentiation [Araki W., et al. Biochem Biophys Res Commun, 1991, 181:265-271; Milward E A, et al. Neuron, 1991, 9:129-137; and Yamamoto K, et al. J Neurobiol, 1994, 25:585-594].

The non-selective serotonin 5HT_{2C} agonist dexnorfenfluramine (DEXNOR) stimulated amyloid precursor protein (APPs) secretion in guinea pigs while reducing levels of Aβ production in vivo following repeat administration [Arjona A, et al. “Effect of a 5HT_{2C} serotonin agonist, dexnorfenfluramine, on amyloid precursor protein metabolism in guinea pigs,” Brain Res, 2002, 951:135-140]. Guinea pigs were chosen because guinea pig and human APP exhibit 98% sequence homology [Beck M, et al. Biochem Biophys Acta, 1997, 1351:17-21], the proteins are processed similarly [Beck M., et al. Neuroscience, 1999, 95:243-254], and the Aβ peptide sequences are identical [Johnstone E M, et al. Brain Res Mol Brain Res, 1991, 10:299-305]. Although DEXNOR is non-selective, the observed effects were attenuated by a selective serotonin 5HT_{2C} antagonist, while a selective serotonin HT_{2A} antagonist did not reverse the DEXNOR effects, indicating the serotonin 5HT_{2C} receptors are the most relevant target for this effect.

In addition, 5-HT stimulates APPs ectodomain secretion via the serotonin 5HT_{2A} and 5HT_{2C} receptors [Nitsch R M, et al. J Biol Chem, 1996, 271(8):4188-4194]. In this study, researchers stimulated 3T3 fibroblasts with serotonin (5-HT), which were stably expressing serotonin 5HT_{2A} or 5HT_{2C} receptors. 5-HT increased APPs secretion in a dose-dependent manner in both cell lines. Maximal stimulation of APPs secretion peaked at about 4-fold. Selective serotonin 5HT_{2A} and 5HT_{2C} antagonists blocked the effects in each cell line.

A serotonin 5HT_{2C} receptor agonist can be effective for treating AD and preventing senile plaques. Support for this claim comes from the fact that Aβ is known to be neurotoxic and a key component in senile plaques involved in AD, APPs secretion and Aβ levels seem to be inversely related, and serotonin 5HT_{2C} agonists increase levels of APPs in vitro in cell lines stably expressing serotonin 5HT_{2C} receptors while in vivo serotonin 5HT_{2C} agonists increase levels of APPs and decrease levels of Aβ as measured in cerebral spinal fluid of guinea pigs.

Evidence exists supporting the use of a compound of the present invention with agonist activity at the serotonin 5HT_{2C} receptor for the treatment of AD. The compound of the invention can be used alone or in combination with another agent or agents (such as but not limited to AChE inhibitors) that are typically prescribed for AD.

Another disease, disorder or condition that can is associated with the function of the 5HT_{2C} receptor is erectile dysfunction (ED). Erectile dysfunction is the inability to achieve or maintain an erection sufficiently rigid for intercourse, ejaculation, or both. An estimated 20-30 million men in the United States have this condition at some time in their lives. The prevalence of the condition increases with age. Five percent of men 40 years of age report ED. This rate increases to between 15% and 25% by the age of 65, and to 55% in men over the age of 75 years.

Erectile dysfunction can result from a number of distinct problems. These include loss of desire or libido, the inability to maintain an erection, premature ejaculation, lack of emission, and inability to achieve an orgasm. Frequently, more than one of these problems presents themselves simultaneously. The conditions may be secondary to other disease states (typically chronic conditions), the result of specific disorders of the urogenital system or endocrine system, secondary to treatment with pharmacological agents (e.g. antihypertensive drugs, antidepressant drugs, antipsychotic drugs, etc.) or the result of psychiatric problems. Erectile dysfunction, when organic, is primarily due to vascular irregularities associated with atherosclerosis, diabetes, and hypertension.

There is evidence for use of a serotonin 5HT_{2C} agonist for the treatment of sexual dysfunction in males and females. The serotonin 5HT_{2C} receptor is involved with the processing and integration of sensory information, regulation of central monoaminergic systems, and modulation of neuroendocrine responses, anxiety, feeding behavior, and cerebrospinal fluid production [Tecott, L. H., et al. Nature 374: 542-546 (1995)]. In addition, the serotonin 5HT_{2C} receptor has been implicated in the mediation of penile erections in rats, monkeys, and humans.

The exact mechanism by which 5HT_{2C} receptors mediate penile erections remains unknown. However, there is good evidence, indirect and direct, supporting the role of serotonin 5HT_{2C} receptors in the mediation of penile erections. Anatomical studies have shown that the penis receives autonomic innervation from sympathetic and parasympathetic nuclei located in the spinal cord [Pescatori E S, et al. J Urol 1993; 149: 627-32]. In agreement, experimental and clinical data support that penile erections are controlled by a spinal reflex. A closer analysis showed that activation of 5HT₂ spinal receptors facilitated pudendal reflex in anesthetized cats [Danuser H and Thor K B, Br J Pharmacol 1996; 118: 150-4]. Accordingly, stimulation of 5HT_{2C} receptors has been shown to be proerectile [Millan M J, et al. European Journal of pharmacology 1997; 325], and 5HT_{2C} receptors have been described on proerectile spinal parasympathetic neurons [Bancila M et al. Neuroscience 1999; 92: 1523-37].

Indirect evidence comes from the research and reports of the side effects induced by the use of selective serotonin reuptake inhibitors (SSRIs). SSRIs have demonstrated antagonist action at the serotonin 5HT_{2C} receptors [Jenck et al. European Journal of Pharmacology 231: 223-229 (1993); Lightlowler et al. European Journal of Pharmacology 296:137-43 (1996); and Palvimaki, E., et al. Psychopharmacology 126: 234-240 (1996)]. Among the most derogatory side effects of SSRIs noted in humans is increased difficulty in attaining penile erection. Although SSRIs have a rich pharmacological profile, it is believed that the antagonist effects of SSRIs at the 5HT_{2C} receptors could be implicated in the inhibition of penile erections [Palvimaki, E., et al. Psychopharmacology 126: 234-240 (1996)].

Further evidence comes from studies with a variety compounds with known agonist activity for the serotonin 5HT_{2C} receptor. Pharmacologic studies with rats and rhesus monkeys provide direct evidence of the proerectile properties of agonist of the serotonin 5-HT_{2C} receptor [Millan M J, et al. European Journal of Pharmacology 1997; 325; and Pomerantz, et al. European Journal of Pharmacology 243:227-34 (1993)]. These pro-erectile effects were unaffected by antagonists for the serotonin 5HT_{2A} and 5HT_{2B} receptors, respectively. Antagonists of the serotonin 5HT_{2C} receptors attenuated the proerectile effects of the 5-HT_{2C} agonists. The inhibition action corresponded to each antagonist's affinity for the 5-HT_{2C} receptors. In addition, agonists of the serotonin 5HT_{2A} and 5HT_{2B} receptors did not elicit penile erections.

In summary, the 5HT_{2C} receptor is a validated and well-accepted receptor target for the prophylaxis and/or treatment of 5HT_{2C} mediated receptor diseases and disorders, such as, obesity, eating disorders, psychiatric disorders, Alzheimer Disease, sexual dysfunction and disorders related thereto. It can be seen that there exists a need for selective 5HT_{2C} receptor agonists that can safely address these needs. The present invention is directed to these, as well as other, important ends.

### SUMMARY OF THE INVENTION

The present invention is drawn to compounds which bind to and activate of the 5HT_{2C} receptor, and uses thereof. The term 5HT_{2C} receptor as used herein includes the human sequences found in GeneBank accession number AF498983, naturally-occurring allelic variants, mammalian orthologs, and recombinant mutants thereof.

One aspect of the present invention pertains to certain N-biaryl and N-arylheteroaryl 2-substituted piperazine derivatives as represented by Formula (Ia):

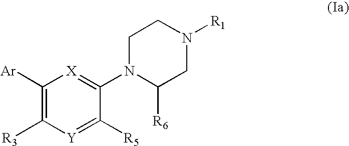

or a pharmaceutically acceptable salt, solvate, or hydrate thereof;
wherein:

X is N or CR₂;

Y is N or CR₄;

R₁ is H or C₁₋₄ alkyl;

R₂, R₃, R₄ and R₅ are each independently H, C₁₋₄ alkyl, C₁₋₄ haloalkyl or halogen provided that at least one group is other than H;

Ar is aryl or heteroaryl optionally substituted with 1, 2, 3, 4 or 5 substituents selected independently from the group consisting of C₁₋₄ acyl, C₁₋₄ acyloxy, C₁₋₄ acylthioxy, C₂₋₄ alkenyl, C₁₋₄ alkoxy, C₁₋₄ alkyl, C₁₋₄ alkylcarboxamido, C₁₋₄ alkylsulfinyl, C₁₋₄ alkylsulfonamide, C₁₋₄ alkylsulfonyl, C₁₋₄ alkylthio, amino, C₁₋₄ alkylamino, carbo-C₁₋₄-alkoxy, carboxamide, cyano, C₂₋₆ dialkylamino, C₁₋₄ haloalkoxy, C₁₋₄ haloalkyl, C₁₋₄ haloalkylsulfinyl, C₁₋₄ haloalkylsulfonyl, C₁₋₄ haloalkylthio, halogen, hydroxyl and thiol; and

R₆ is C₁₋₄ alkyl;

provided that when X and Y are both CH, R₁ and R₃ are both H, R₅ is F and R₆ is methyl, then Ar is a group other than phenyl.

Another aspect of the present invention pertains to pharmaceutical compositions comprising a compound of the present invention in combination with a pharmaceutically acceptable carrier.

Another aspect of the present invention pertains to methods of activating a 5HT_{2C} receptor comprising contacting the receptor with a therapeutically effective amount of a compound of the present invention. In some embodiments, the compound is an agonist of the 5HT_{2C} receptor.

Another aspect of the present invention pertains to methods of treating a 5HT_{2C} receptor associated disorder comprising administering to an individual in need of such treatment an effective amount of a compound of the present invention or a pharmaceutical composition thereof.

Another aspect of the present invention pertains to methods of treating a disorder of the central nervous system; damage to the central nervous system; cardiovascular disorders; gastrointestinal disorders; diabetes insipidus or sleep apnea comprising administering to an individual in need of such treatment a therapeutically effective amount of a compound of the present invention or a pharmaceutical composition thereof.

Another aspect of the present invention pertains to methods of decreasing food intake of an individual comprising administering to the individual a therapeutically effective amount of a compound of the present invention or a pharmaceutical composition thereof.

Another aspect of the present invention pertains to methods of inducing satiety in an individual comprising administering to the individual a therapeutically effective amount of a compound of the present invention or a pharmaceutical composition thereof.

Another aspect of the present invention pertains to methods of controlling weight gain of an individual comprising administering to the individual suffering from weight control a therapeutically effective amount of a compound of the present invention or a pharmaceutical composition thereof.

Another aspect of the present invention pertains to methods of producing pharmaceutical compositions comprising admixing a compound of the present invention and a pharmaceutically acceptable carrier.

Another aspect of the present invention pertains to compounds of the present invention for use in methods of treatment of the human or animal body by therapy.

Another aspect of the present invention pertains to compounds of the present invention for use in methods of treatment of a 5HT_{2C} receptor associated disorder of the human or animal body by therapy.

Another aspect of the present invention pertains to compounds of the present invention for the production of a medicament for use in the treatment of a 5HT_{2C} receptor associated disorder.

Another aspect of the present invention pertains to use of compounds of the present invention for the production of a medicament for use in the treatment of a disorder of the central nervous system; damage to the central nervous system; cardiovascular disorders; gastrointestinal disorders; diabetes insipidus or sleep apnea.

In some embodiments, the disorder of the central nervous system is selected from the group consisting of depression, atypical depression, bipolar disorders, anxiety disorders, obsessive-compulsive disorders, social phobias or panic states, sleep disorders, sexual dysfunction, psychoses, schizophrenia, migraine and other conditions associated with cephalic pain or other pain, raised intracranial pressure, epilepsy, personality disorders, Alzheimer disease, age-related behavioral disorders, behavioral disorders associated with dementia, organic mental disorders, mental disorders in childhood, aggressivity, age-related memory disorders, chronic fatigue syndrome, drug and alcohol addiction, obesity, bulimia, anorexia nervosa and premenstrual tension.

In some embodiments, the disorder of the central nervous system is obesity.

In some embodiments, the disorder of the central nervous system is Alzheimer disease.

In some embodiments, the disorder of the central nervous system is Male erectile dysfunction.

In some embodiments, the damage to the central nervous system is by trauma, stroke, neurodegenerative diseases, toxic CNS diseases or infective CNS diseases.

In some embodiments, the damage to the central nervous system is by encephalitis or meningitis.

In some embodiments, the cardiovascular disorder is thrombosis.

In some embodiments, the gastrointestinal disorder is dysfunction of gastrointestinal motility.

In some embodiments, the individual is a mammal. In some embodiments, the mammal is a human. In some embodiments, the human has a body mass index of about 18.5 to about 45. In some embodiments, the human has a body mass index of about 25 to about 45. In some embodiments, the human has a body mass index of about 30 to about 45. In some embodiments, the human has a body mass index of about 35 to about 45.

### BRIEF DESCRIPTION OF THE FIGURES

In the illustrated syntheses outlined in the Figures, the labeled substituents have the same identifications as set out in the definitions of the compounds of the present invention of Formula (Ia) and the Formulae of the subgenera as described herein.

FIG. 1 shows two general synthetic schemes for the preparation of intermediates and compounds of the present invention. One route to compounds of the invention includes coupling an optionally substituted 1,3-dihalobenzene with an aryl or heteroaryl boronic acid and subsequently with a mono-protected 2-substituted piperazine under suitable coupling conditions known in the art. PG represents a protecting group wherein one particularly useful group is the BOC group. The protecting group is removed to provide compounds of the present invention wherein R₁ is H. Another route to compounds of the invention includes coupling an optionally substituted 1,3-dihalobenzene with a mono-protected 2-substituted piperazine, deprotecting and subsequently further coupling with an aryl or heteroaryl boronic acid. It is understood that similar coupling methods known in the art can also be used for the various couplings, and Halo includes, for example, I, Br and Cl, wherein Halo can be replaced with a triflate group under certain synthetic conditions. In a subsequent step, the piperazine (when R₁ is H) can be alkylated to introduce R₁ as an alkyl group; an alternative method for introducing the alkyl group for R₁ is shown in FIG. 2.

FIG. 2 shows two general synthetic schemes for the preparation of intermediates and compounds of the present invention wherein R₁ is an alkyl group. The routes shown in FIG. 2 are similar to those described in FIG. 1 except that the protecting group is replaced with R₁.

### DETAILED DESCRIPTION OF THE INVENTION

### Definitions

For clarity and consistency, the following definitions will be used throughout this patent document.

AGONISTS shall mean moieties that interact and activate the receptor, such as the 5HT_{2c} receptor and initiates a physiological or pharmacological response characteristic of that receptor. For example, when moieties activate the intracellular response upon binding to the receptor, or enhance GTP binding to membranes.

The term ANTAGONISTS is intended to mean moieties that competitively bind to the receptor at the same site as agonists (for example, the endogenous ligand), but which do not activate the intracellular response initiated by the active form of the receptor, and can thereby inhibit the intracellular responses by agonists or partial agonists.

Chemical Group, Moiety or Radical:

The term “C₁₋₄ acyl” denotes an alkyl radical attached to a carbonyl wherein the definition of alkyl has the same definition as described herein; some examples include formyl, acetyl, propionyl, butanoyl, iso-butanoyl, and the like.

The term “C₁₋₄ acyloxy” denotes an acyl radical attached to an oxygen atom wherein acyl has the same definition has described herein; some examples include acetyloxy, propionyloxy, butanoyloxy, iso-butanoyloxy and the like.

The term “C₁₋₄ acylthioxy” denotes a thioacyl [i.e., alkyl-C(═S)—] radical attached to an oxygen atom; some examples include acetylthiooxy [i.e., CH₃C(═S)O—], propionylthiooxy, iso-butanoylthiooxy and the like.

The term “C₂₋₄ alkenyl” denotes a radical containing 2 to 4 carbons wherein at least one carbon-carbon double bond is present, some embodiments have 3 carbons, and some embodiments have 2 carbons. Both E and Z isomers and mixtures of E and Z isomers are embraced by the term “alkenyl.” Examples of an alkenyl include vinyl, allyl, 2-butenyl, 3-butenyl, and the like.

The term “C₁₋₄ alkoxy” as used herein denotes a radical alkyl, as defined herein, attached directly to an oxygen atom. Examples include methoxy, ethoxy, n-propoxy, iso-propoxy, n-butoxy, t-butoxy, iso-butoxy and the like.

The term “C₁₋₄ alkyl” denote a straight or branched carbon radical containing 1 to 8 carbons or 1 to 4 carbons respectively, some embodiments are 1 to 6 carbons, some embodiments are 1 to 3 carbons, and some embodiments are 1 or 2 carbons. Examples of an alkyl include methyl, ethyl, n-propyl, iso-propyl, n-butyl, iso-butyl, t-butyl, sec-butyl, n-pentyl, iso-pentyl, sec-pentyl, neo-pentyl, pent-3-yl, 2-methyl-but-1-yl, 1,2-dimethyl-prop-1-yl, n-hexyl, iso-hexyl, sec-hexyl, neo-hexyl, 1-ethyl-2-methyl-prop-1-yl, 1,2,2-trimethyl-prop-1-yl, 1,1,2-trimethyl-prop-1-yl, 1-ethyl-1-methyl-prop-1-yl, 1,1-dimethyl-but-1-yl, 1,2-dimethyl-but-1-yl, 2,3-dimethyl-but-1-yl, 2,2-dimethyl-but-1-yl, 1,3-dimethyl-but-1-yl, hex-3-yl, 2-methyl-pent-1-yl, 3-methyl-pent-1-yl, and the like.

The term “C₁₋₄ alkylcarboxamido” denotes a single alkyl group attached to an amide, wherein alkyl has the same definition as found herein. The C₁₋₅ alkylcarboxamido may be represented by the following:

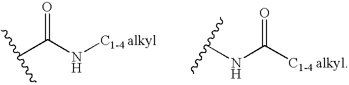

The term “C₁₋₄ alkylsulfinyl” denotes an alkyl radical attached to a sulfoxide radical of the formula: —S(O)— wherein the alkyl radical has the same definition as described herein. Examples include methylsulfinyl, ethylsulfinyl and the like.

The term “C₁₋₄ alkylsulfonamide” refers to the groups

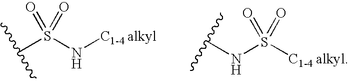

The term “C₁₋₄ alkylsulfonyl” denotes an alkyl radical attached to a sulfone radical of the formula: —S(O)₂— wherein the alkyl radical has the same definition as described herein. Examples include methylsulfonyl, ethylsulfonyl and the like.

The term “C₁₋₄ alkylthio” denotes an alkyl radical attached to a sulfide of the formula: —S—wherein the alkyl radical has the same definition as described herein. Examples include methylsulfanyl (i.e., CH₃S—), ethylsulfanyl, isopropylsulfanyl and the like.

The term “C₁₋₄ alkylamino” denotes one alkyl radical attached to an amino radical wherein the alkyl radical has the same meaning as described herein. Some examples include methylamino, ethylamino, propylamino and the like.

The term “aryl” denotes an aromatic ring radical containing 6 to 10 ring carbons. Examples include phenyl and naphthyl.

The term “carbo-C₁₋₄-alkoxy” refers to an alkyl ester of a carboxylic acid, wherein the alkyl group is C₁₋₄. Examples include carbomethoxy, carboethoxy, carboisopropoxy and the like.

The term “carboxamide” refers to the group —CONH₂.

The term “cyano” denotes the group —CN.

The term “C₂₋₆ dialkylamino” denotes an amino substituted with two of the same or different C₁₋₃ alkyl radicals wherein alkyl radical has the same definition as described herein. Some examples include dimethylamino, methylethylamino, diethylamino and the like.

The term “C₁₋₄ haloalkoxy” denotes a haloalkyl, as defined herein, that is directly attached to an oxygen to form a difluoromethoxy, trifluoromethoxy, 2,2,2-trifluoroethoxy, pentafluoroethoxy and the like.

The term “amino” denotes the group —NH₂.

The term “C₁₋₄ haloalkyl” denotes an alkyl group, defined herein, wherein the alkyl is substituted with at least one halogen up to fully substituted represented by the formula CₙL₂ₙ₊₁, wherein L is a halogen; when more than one halogen is present then they may be the same or different and selected from F, Cl, Br or I. Examples include fluoromethyl, difluoromethyl, trifluoromethyl, chlorodifluoromethyl, 2,2,2-trifluoroethyl, pentafluoroethyl and the like.

The term “C₁₋₄ haloalkylsulfinyl” denotes a haloalkyl radical attached to a sulfoxide of the formula: —S(O)— wherein the alkyl radical has the same definition as described herein. Examples include trifluoromethylsulfinyl, 2,2,2-trifluoroethylsulfinyl, 2,2-difluoroethylsulfinyl and the like.

The term “C₁₋₄ haloalkylsulfonyl” denotes a haloalkyl attached to a sulfone of the formula: —S(O)₂— wherein haloalkyl has the same definition as described herein. Examples include trifluoromethylsulfonyl, 2,2,2-trifluoroethylsulfonyl, 2,2-difluoroethylsulfonyl and the like.

The term “C₁₋₄ haloalkylthio” denotes an alkylthio radical substituted with one or more halogens. Examples include trifluoromethylthio, 1,1-difluoroethylthio, 2,2,2-trifluoroethylthio and the like.

The term “halogen” or “halo” denotes F, Cl, Br and I.

The term “heteroaryl” denotes an aromatic ring system that may be a single ring, two fused rings or three fused rings wherein at least one ring carbon is replaced with a heteroatom selected from, but are not limited to, the group consisting of O, S and N wherein the N can be optionally substituted with H, C₁₋₄ acyl or C₁₋₄ alkyl. Examples of heteroaryl groups include, but are not limited to, pyridyl, benzofuranyl, pyrazinyl, pyridazinyl, pyrimidinyl, triazinyl, quinoline, benzoxazole, benzothiazole, 1H-benzimidazole, isoquinoline, quinazoline, quinoxaline and the like. In some embodiments, the heteroaryl atom is selected from the group O, S, NH and N; examples include, but are not limited to, pyrrole, indole, and the like.

The term “hydroxyl” refers to the group —OH.

The term “thiol” denotes the group —SH.

COMPOSITION shall mean a material comprising at least two compounds or two components; for example, and not limitation, a Pharmaceutical Composition is a Composition.

CONTACT or CONTACTING shall mean bringing the indicated moieties together, whether in an in vitro system or an in vivo system. Thus, “contacting” a 5HT_{2C} receptor with a compound of the invention includes the administration of a compound of the present invention to an individual, preferably a human, having a 5HT_{2C} receptor, as well as, for example, introducing a compound of the invention into a sample containing a cellular or more purified preparation containing a 5HT_{2C} receptor.

IN NEED OF TREATMENT as used herein refers to a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

INDIVIDUAL as used herein refers to any animal, including mammals, preferably mice, rats, other rodents, rabbits, dogs, cats, swine, cattle, sheep, horses, or primates, and most preferably humans.

PHARMACEUTICAL COMPOSITION shall mean a composition comprising at least one active ingredient, whereby the composition is amenable to investigation for a specified, efficacious outcome in a mammal (for example, but not limited to, a human). Those of ordinary skill in the art will understand and appreciate the techniques appropriate for determining whether an active ingredient has a desired efficacious outcome based upon the needs of the artisan.

THERAPEUTICALLY EFFECTIVE AMOUNT as used herein refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) Preventing the disease; for example, preventing a disease, condition or disorder in an individual that may be predisposed to the disease, condition or disorder but does not yet experience or display the pathology or symptomatology of the disease,

(2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and

(3) Ameliorating the disease; for example, ameliorating a disease, condition or disorder in an individual that is experiencing or displaying the pathology or symptomatology of the disease, condition or disorder (i.e., reversing the pathology and/or symptomatology).

### Compounds of the Invention

One aspect of the present invention pertains to certain N-biaryl and N-arylheteroaryl 2-substituted piperazine derivatives as represented by Formula (Ia):

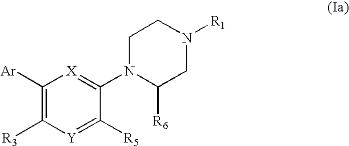

or a pharmaceutically acceptable salt thereof; wherein Ar, X, Y, R₁, R₃, R₅, and R₆ have the same definitions as described herein, supra and infra.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables (e.g., Ar, X, Y, R₁, R₃, R₅, R₆, etc.) contained within the generic chemical formulae described herein [e.g. (Ia), (Ie), (Ie), etc.] are specifically embraced by the present invention just as if they were explicitly disclosed, to the extent that such combinations embrace compounds that result in stable compounds (ie., compounds that can be isolated, characterized and tested for biological activity). In addition, all subcombinations of the chemical groups listed in the embodiments describing such variables, as well as all subcombinations of uses and medical indications described herein, are also specifically embraced by the present invention just as if each of such subcombination of chemical groups and subcomination of uses and medical indications were explicitly disclosed herein.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

It is understood and appreciated that compounds of Formula (Ia) may have one or more chiral centers, and therefore can exist as enantiomers and/or diastereomers. The invention is understood to extend to and embrace all such enantiomers, diastereomers and mixtures thereof, including but not limited to racemates. Accordingly, one embodiment of the present invention pertains to compounds of Formula (Ia) and formulae used throughout this disclosure that are R enantiomers. Further, one embodiment of the present invention pertains to compounds of Formula (Ia) and formulae used throughout this disclosure that are S enantiomers. It is understood that compounds of Formula (Ia) and formulae used throughout this disclosure are intended to represent all individual enantiomers and mixtures thereof, unless stated or shown otherwise.

Some embodiments of the present invention pertain to compounds wherein X is N. Some embodiments can be represented by Formula (Ic) as illustrated below:

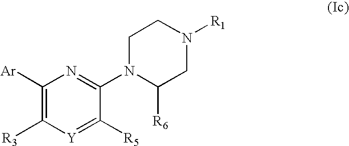

wherein each variable in Formula (Ic) has the same meaning as described herein, supra and infra.

Some embodiments of the present invention pertain to compounds wherein X is CR₂. Some embodiments can be represented by Formula (Ie) as illustrated below:

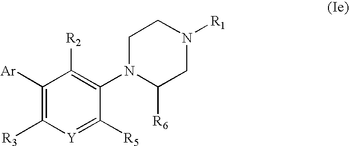

wherein each variable in Formula (Ie) has the same meaning as described herein, supra and infra.

Some embodiments of the present invention pertain to compounds wherein Y is N. Some embodiments can be represented by Formula (Ig) as illustrated below:

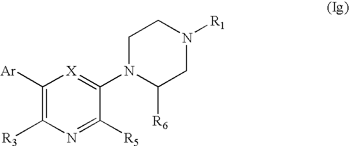

wherein each variable in Formula (Ig) has the same meaning as described herein, supra and infra.

Some embodiments of the present invention pertain to compounds wherein Y is CR₄. Some embodiments can be represented by Formula (Ii) as illustrated below:

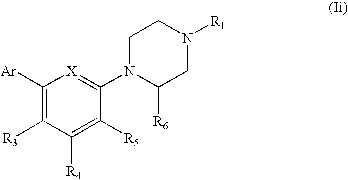

wherein each variable in Formula (Ii) has the same meaning as described herein, supra and infra.

Some embodiments of the present invention pertain to compounds wherein X is CR₂; and Y is CR₄. Some embodiments can be represented by Formula (Ik) as illustrated below:

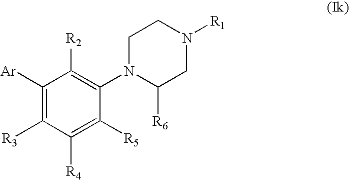

wherein each variable in Formula (Ik) has the same meaning as described herein, supra and infra.

Some embodiments of the present invention pertain to compounds wherein R₁ is H. Some embodiments can be represented by Formula (Im) as illustrated below:

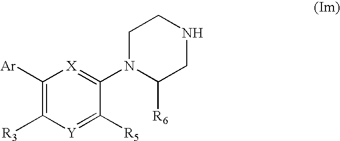

wherein each variable in Formula (Im) has the same meaning as described herein, supra and infra.

Some embodiments of the present invention pertain to compounds wherein R₁ is C₁₋₄ alkyl.

Some embodiments of the present invention pertain to compounds wherein R₁ is methyl.

Some embodiments of the present invention pertain to compounds wherein R₂ is halogen.

Some embodiments of the present invention pertain to compounds wherein R₂ is F.

Some embodiments of the present invention pertain to compounds wherein R₂ is CF₃.

Some embodiments of the present invention pertain to compounds wherein R₃ is halogen.

Some embodiments of the present invention pertain to compounds wherein R₃ is F.

Some embodiments of the present invention pertain to compounds wherein R₃ is CF₃.

Some embodiments of the present invention pertain to compounds wherein R₄ is halogen.

Some embodiments of the present invention pertain to compounds wherein R₄ is F.

Some embodiments of the present invention pertain to compounds wherein R₄ is CF₃.

Some embodiments of the present invention pertain to compounds wherein R₅ is halogen.

Some embodiments of the present invention pertain to compounds wherein R₅ is F.

Some embodiments of the present invention pertain to compounds wherein R₅ is CF₃.

Some embodiments of the present invention pertain to compounds wherein X is CR₂; Y is CR₄; R₂ is halogen; and R₃, R₄ and R₅ are each H.

Some embodiments of the present invention pertain to compounds wherein X is CR₂; Y is CR₄; R₂ is F; and R₃, R₄ and R₅ are each H.

Some embodiments of the present invention pertain to compounds as illustrated below:

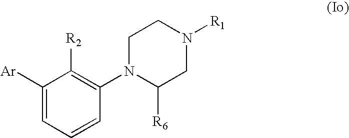

wherein Ar, R₁, R₂ and R₆ in Formula (Io) have the same meaning as described herein, supra and infra.

Some embodiments of the present invention pertain to compounds wherein X is CR₂; Y is CR₄; R₃ is halogen; and R₂, R₄ and R₅ are each H.

Some embodiments of the present invention pertain to compounds wherein X is CR₂; Y is CR₄; R₃ is F; and R₂, R₄ and R₅ are each H.

Some embodiments of the present invention pertain to compounds as illustrated below:

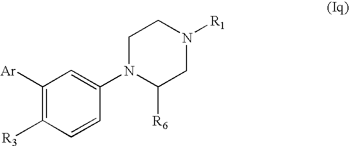

wherein Ar, R₁, R₃ and R₆ in Formula (Iq) have the same meaning as described herein, supra and infra.

Some embodiments of the present invention pertain to compounds wherein X is CR₂; Y is CR₄; R₄ is halogen; and R₂, R₃ and R₅ are each H.

Some embodiments of the present invention pertain to compounds wherein X is CR₂; Y is CR₄; R₄ is F; and R₂, R₃ and R₅ are each H.

Some embodiments of the present invention pertain to compounds as illustrated below:

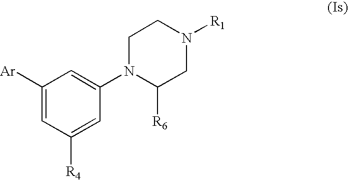

wherein Ar, R₁, R₄ and R₆ in Formula (Is) have the same meaning as described herein, supra and infra.

Some embodiments of the present invention pertain to compounds wherein X is CR₂; Y is N; and R₂, R₃ and R₅ are each H.

Some embodiments of the present invention pertain to compounds wherein X is N; Y is CR₄; and R₃, R₄ and R₅ are each H.

Some embodiments of the present invention pertain to compounds wherein Ar is selected from the group consisting of thienyl, pyridinyl, phenyl and furanyl.

Some embodiments of the present invention pertain to compounds wherein Ar is selected from the group consisting of thiophen-3-yl, pyridine-3-yl, phenyl and furan-3-yl.

Some embodiments of the present invention pertain to compounds wherein Ar is selected from the group consisting of Ar is selected from the group consisting of thiophen-3-yl, pyridine-3-yl, phenyl, 2-fluorophenyl, furan-3-yl, 3-fluorophenyl, 4-fluorophenyl, and 2,6-difluorophenyl.

Some embodiments of the present invention pertain to compounds wherein R₆ is methyl.

Some embodiments of the present invention pertain to compounds wherein:

R₁ is H;

X is N or CR₂; and Y is N or CR₄; provided that not both X and Y are N; R₂, R₃, R₄ and R₅ are each independently H or halogen provided that at least one group is halogen;

Ar is selected from the group consisting of thienyl, pyridinyl, phenyl and furanyl; and

R₆ is methyl.

Some embodiments of the present invention pertain to compounds wherein:

R₁ is H;

X is N or CR₂; and Y is N or CR₄; provided that not both X and Y are N;

R₂, R₃, R₄ and R₅ are each independently H or halogen provided that at least one group is halogen;

Ar is selected from the group consisting of thienyl, pyridinyl, phenyl and furanyl, each optionally substituted with 1 or 2 halo groups; and

R₆ is methyl.

Some embodiments of the present invention pertain to compounds wherein:

R₁ is H;

X is N;

Y is CR₄;

R₃, R₄ and R₅ are each H;

Ar is selected from the group consisting of thiophen-3-yl, pyridine-3-yl, phenyl and furan-3-yl; and

R₆ is methyl.

Some embodiments of the present invention pertain to compounds wherein:

R₁ is H;

X is CR₂;

Y is N;

R₂, R₃ and R₅ are each H;

Ar is selected from the group consisting of thiophen-3-yl, pyridine-3-yl, phenyl and furan-3-yl; and

R₆ is methyl.

Some embodiments of the present invention pertain to compounds wherein:

R₁ is H;

X is CR₂;

Y is CR₄;

R₂, R₃, R₄ and R₅ are each independently H or halogen provided that at least one group is halogen;

Ar is selected from the group consisting of thiophen-3-yl, pyridine-3-yl, phenyl and furan-3-yl; and

R₆ is methyl.

Some embodiments of the present invention pertain to compounds wherein:

R₁ is H;

X is CR₂;

Y is CR₄;

R₂, R₃, R₄ and R₅ are each independently H or halogen provided that at least one group is halogen;

Ar is selected from the group consisting of thiophen-3-yl, pyridine-3-yl, phenyl, 2-fluorophenyl, furan-3-yl, 3-fluorophenyl, 4-fluorophenyl, and 2,6-difluorophenyl; and

R₆ is methyl.

Some embodiments of the present invention pertain to compounds as represented in TABLE 2 below. Some embodiments of the present invention include every combination of one or more compounds selected from the compound in TABLE 2.

TABLE 2

| Cmpd | | |
|---|---|---|
| No. | Chemical Structure | Chemical Name |
| 1 | 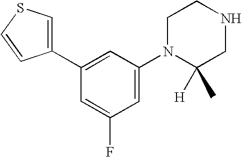 | (R)-1-(3-Fluoro-5-thiophen-2-yl-phenyl)-2-methyl-piperazine |
| 2 | 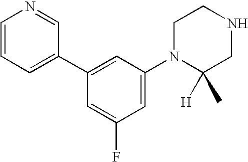 | (R)-1-(3-Fluoro-5-pyridin-3-yl-phenyl)-2-methyl-piperazine |
| 3 | 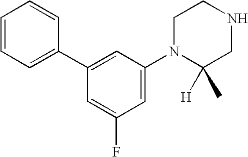 | (R)-1-(5-Fluoro-biphenyl-3-yl)-2-methyl-piperazine |
| 4 | 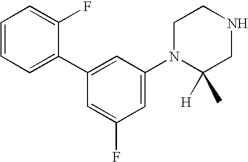 | (R)-1-(5,2′-Difluoro-biphenyl-3-yl)-2-methyl-piperazine |
| 5 | 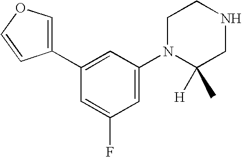 | (R)-1 -(3-Fluoro-5-furan-3-yl-phenyl)-2-methyl-piperazine |
| 6 | 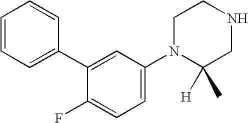 | (R)-1-(6-Fluoro-biphenyl-3-yl)-2-methyl-piperazine |
| 7 | 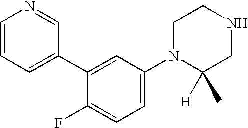 | (R)-1-(4-Fluoro-3-pyridin-3-yl-phenyl)-2-methyl-piperazine |
| 8 | 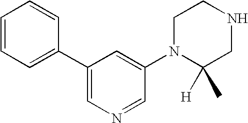 | (R)-2-Methyl-1-(5-phenylpyridin-3-yl)-piperazine |
| 9 | 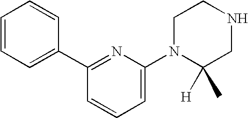 | (R)-2-Methyl-1-(6-phenylpyridin-2-yl)-piperazine |
| 10 | 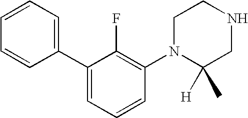 | (R)-1-(2-Fluoro-biphenyl-3-yl)-2-methyl-piperazine |
| 11 | 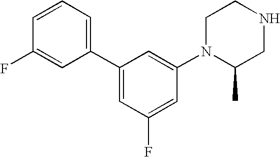 | (R)-1-(5,3′-Difluoro-biphenyl-3-yl)-2-methyl-piperazine |
| 12 | 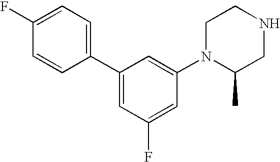 | (R)-1-(5,4′-Difluoro-biphenyl-3-yl)-2-methyl-piperazine |
| 13 | 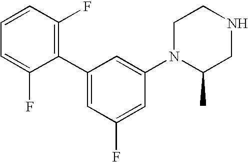 | (R)-2-Methyl-1-(5,2′,6′-trifluoro-biphenyl-3-yl)-piperazine |
| 14 | 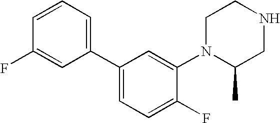 | (R)-1-(4,3′-Difluoro-biphenyl-3-yl)-2-methyl-piperazine |
| 15 | 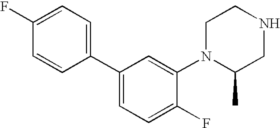 | (R)-1-(4,4′-Difluoro-biphenyl-3-yl)-2-methyl-piperazine |
| 16 | 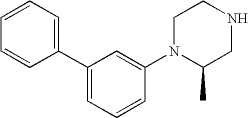 | (R)-1-Biphenyl-3-yl-2-methyl-piperazine |

Additionally, individual compounds and chemical genera of the present invention, such as Formula (Ia) and related Formulae therefrom, encompass all pharmaceutically acceptable salts, solvates, and particularly hydrates, thereof.

It is understood that the present invention embraces racemic mixtures of compounds disclosed herein. Accordingly, some embodiments of the present invention include every combination of one or more compounds selected from the following group:

1-(3-Fluoro-5-thiophen-3-yl-phenyl)-2-methyl-piperazine; 1-(3-Fluoro-5-pyridin-3-yl-phenyl)-2-methyl-piperazine; 1-(5-Fluoro-biphenyl-3-yl)-2-methyl-piperazine; 1-(5,2′-Difluoro-biphenyl-3-yl)-2-methyl-piperazine; 1-(3-Fluoro-5-furan-3-yl-phenyl)-2-methyl-piperazine; 1-(6-Fluoro-biphenyl-3-yl)-2-methyl-piperazine; 1-(4-Fluoro-3-pyridin-3-yl-phenyl)-2-methyl-piperazine; 2-Methyl-1-(5-phenyl-pyridin-3-yl)-piperazine; 2-Methyl-1-(6-phenyl-pyridin-2-yl)-piperazine; 1-(2-Fluoro-biphenyl-3-yl)-2-methyl-piperazine; 1-(5,3′-Difluoro-biphenyl-3-yl)-2-methyl-piperazine; 1-(5,4′-Difluoro-biphenyl-3-yl)-2-methyl-piperazine; 2-Methyl-1-(5,2′,6′-trifluoro-biphenyl-3-yl)-piperazine; 1-(4,3′-Difluoro-biphenyl-3-yl)-2-methyl-piperazine; 1-(4,4′-Difluoro-biphenyl-3-yl)-2-methyl-piperazine; and 1-Biphenyl-3-yl-2-methyl-piperazine.

It is understood that the present invention embraces each diastereomer, each enantiomer and mixtures thereof of each compound and generic Formulae disclosed herein just as if they were each individually disclosed with the specific stereochemical designation for each chiral atom, for example carbon. Accordingly, some embodiments of the present invention include every combination of one or more compounds selected from the following group:

(S)-1-(3-Fluoro-5-thiophen-3-yl-phenyl)-2-methyl-piperazine; (S)-1-(3-Fluoro-5-pyridin-3-yl-phenyl)-2-methyl-piperazine; (S)-1-(5-Fluoro-biphenyl-3-yl)-2-methyl-piperazine; (S)-1-(5,2′-Difluoro-biphenyl-3-yl)-2-methyl-piperazine; (S)-1-(3-Fluoro-5-furan-3-yl-phenyl)-2-methyl-piperazine; (S)-1-(6-Fluoro-biphenyl-3-yl)-2-methyl-piperazine; (S)-1-(4-Fluoro-3-pyridin-3-yl-phenyl)-2-methyl-piperazine; (S)-2-Methyl-1-(5-phenyl-pyridin-3-yl)-piperazine; (S)-2-Methyl-1-(6-phenyl-pyridin-2-yl)-piperazine; (S)-1-(2-Fluoro-biphenyl-3-yl)-2-methyl-piperazine; (S)-1-(5,3′-Difluoro-biphenyl-3-yl)-2-methyl-piperazine; (S)-1-(5,4′-Difluoro-biphenyl-3-yl)-2-methyl-piperazine; (S)-2-Methyl-1-(5,2′,6′-trifluoro-biphenyl-3-yl)-piperazine; (S)-1-(4,3′-Difluoro-biphenyl-3-yl)-2-methyl-piperazine; (S)-1-(4,4′-Difluoro-biphenyl-3-yl)-2-methyl-piperazine; and (S)-1-Biphenyl-3-yl-2-methyl-piperazine.

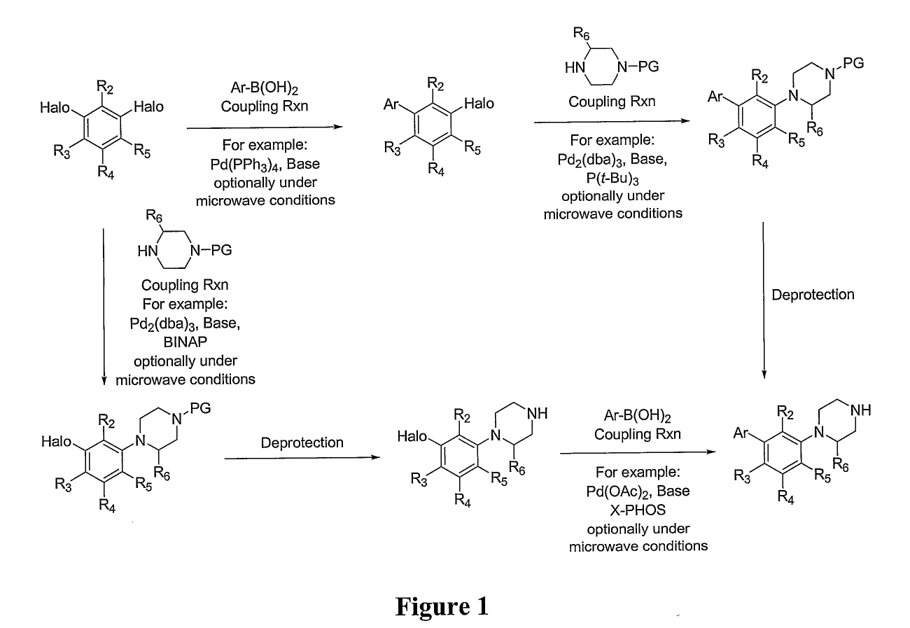

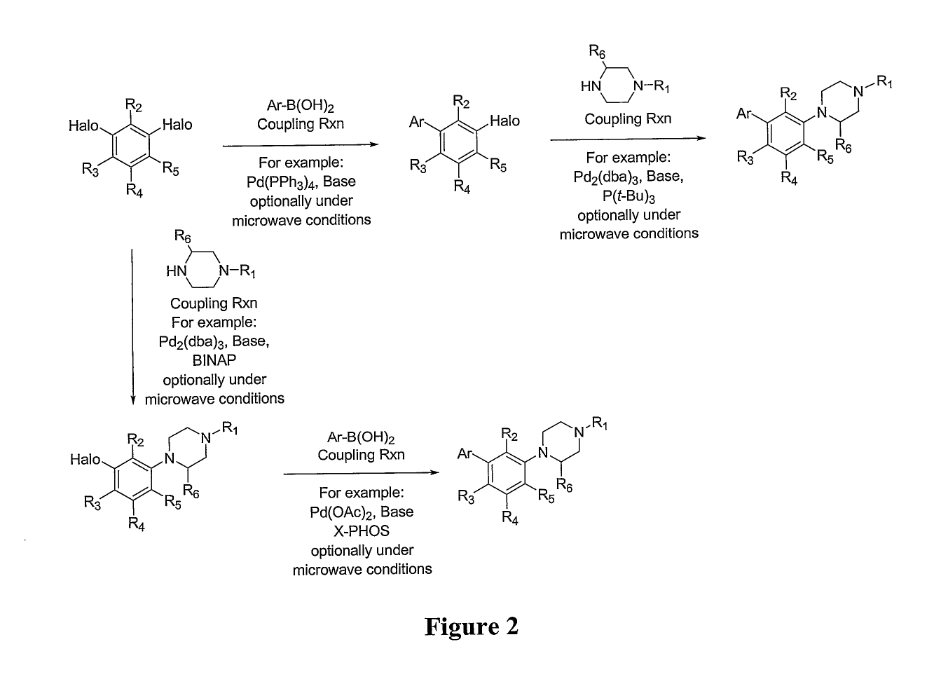

The compounds of the Formula (Ia) of the present invention can be prepared according to the general synthetic schemes in FIGS. 1 and 2 as well as relevant published literature procedures that are used by one skilled in the art. Exemplary reagents and procedures for these reactions appear hereinafter in the working Examples. Protection and deprotection may be carried out by procedures generally known in the art (see, for example, Greene, T. W. and Wuts, P. G. M., Protecting Groups in Organic Synthesis, 3^{rd} Edition, 1999 [Wiley]; incorporated herein by reference in its entirity).

The present invention also encompasses diastereomers as well as optical isomers, e.g. mixtures of enantiomers including racemic mixtures, as well as individual enantiomers and diastereomers, which arise as a consequence of structural asymmetry in certain compounds of the invention. Separation of the individual isomers or selective synthesis of the individual isomers is accomplished by application of various methods which are well known to practitioners in the art, such as chiral HPLC to separate enantiomers.

At various places in the present specification substituents present as a part of the compounds of the invention are disclosed in groups or in ranges. It is specifically intended that the invention include each and every individual subcombination of the members of such groups and ranges. For example, the term “C₁₋₄ alkyl” is specifically intended to individually and separately disclose methyl, ethyl, C₃ alkyl and C₄ alkyl.

### Methods and Use:

One aspect of the present invention pertains to methods of activating a 5HT_{2C} receptor comprising contacting the receptor with a therapeutically effective amount or dose of a compound as described herein. Preferably, compounds of the present invention are agonists of the 5HT_{2C} receptor.

Another aspect of the present invention pertains to methods of treatment of a 5HT_{2C} receptor associated disease in an individual comprising administering to the individual in need of such treatment a therapeutically effective amount or dose of a compound of the present invention or a pharmaceutical composition thereof. In some embodiments, the 5HT_{2C} receptor associated disease is selected from the group consisting of disorders of the central nervous system; damage to the central nervous system; cardiovascular disorders; gastrointestinal disorders; diabetes insipidus and sleep apnea. In some embodiments, the individual is a mammal. Preferably, the mammal is a human.

In some embodiments, the 5HT_{2C} receptor associated related disease is selected from the group consisting of depression, atypical depression, bipolar disorders, anxiety, anxiety disorders, obsessive-compulsive disorders, social phobias, panic states, attention deficit hyperactivity disorder, disruptive behavior disorders, impulse control disorders, borderline personality disorder, sleep disorders (e.g., sleep apnea), autism, seizure disorders, mutism, selective mutism, childhood anxiety disorders, sexual dysfunction in males (e.g., premature ejaculation and erectile difficulty or dysfunction), sexual dysfunction in females, psychoses, schizophrenia, migraine and other conditions associated with cephalic pain or other pain, raised intracranial pressure, epilepsy, personality disorders, Alzheimer disease, age-related behavioral disorders, behavioral disorders associated with dementia, dementia of aging, organic mental disorders, mental disorders in childhood, aggressivity, age-related memory disorders, memory loss, chronic fatigue syndrome, drug and alcohol addiction, alcoholism, tobacco abuse, weight loss, obesity, bulimia, bulimia nervosa, anorexia nervosa, binge eating disorder, premenstrual tension, premenstrual syndrome (PMS or late luteal phase dysphoric disorder), post-traumatic syndrome, spinal cord injury, damage of the central nervous system (e.g., trauma, stroke, neurodegenerative diseases or toxic or infective disorders (e.g., thrombosis), gastrointestinal disorders (e.g., dysfunction of gastrointestinal motility), diabetes insipidus, and type II diabetes.

In some embodiments, the 5HT_{2C} receptor associated disease is selected from the group consisting of high blood pressure, hypertension, high blood cholesterol, dyslipidemia, type II (non-insulin dependent) diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence), uric acid nephrolithiasis, psychological disorders (such as depression, eating disorders, distorted body image, and low self esteem).

In some embodiments, the 5HT_{2C} receptor associated disease is selected from the group consisting of psychiatric symptoms and behaviors in individuals with eating disorders such as, but not limited to, anorexia nervosa and bulimia nervosa. Individuals with eating disorders often demonstrate social isolation. For example, anorexic individuals often present symptoms of being depressed, anxious, obsession, perfectionistic traits, and rigid cognitive styles as well as sexual disinterest. In addition to anorexia nervosa and bulimia nervosa, other eating disorders include, binge eating disorder (compulsive eating) and ED-NOS (i.e., eating disorders not otherwise specified—an official diagnosis). An individual diagnosed with ED-NOS possess atypical eating disorders including situations in which the individual meets all but a few of the criteria for a particular diagnosis. In essence, what the individual is doing with regard to food and weight is neither normal nor healthy.

In some embodiments, the 5HT_{2C} receptor associated disease is selected from the group consisting of anorexia athletica (compulsive exercising), body dysmorphic disorder (bigorexia), infection-triggered auto immune subtype of anorexia in children, orthorexia nervosa, night-eating syndrome, nocturnal sleep-related eating disorder, rumination syndrome, gourmand syndrome, Prader-Willi syndrome, pica, and cyclic vomiting syndrome.

Another aspect of the present invention pertains to methods of decreasing food intake of an individual comprising administering to the individual a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In farther embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of inducing satiety in an individual comprising administering to the individual a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of controlling weight gain of an individual comprising administering to the individual suffering from weight control a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of producing a pharmaceutical composition comprising admixing at least one compound of the present invention and at least one pharmaceutically acceptable carrier.

Another aspect of the present invention pertains to compounds, as described herein, for use in a method of treatment of disorders of the central nervous system; damage to the central nervous system; cardiovascular disorders; gastrointestinal disorders; diabetes insipidus or sleep apnea of the human or animal body by therapy.

Another aspect of the present invention pertains to use of compounds, as described herein, for the production of a medicament for use in the treatment or prophylaxis of disorders of the central nervous system; damage to the central nervous system; cardiovascular disorders; gastrointestinal disorders; diabetes insipidus or sleep apnea.

In some embodiments, the disorders of the central nervous system are selected the group consisting of depression, atypical depression, bipolar disorders, anxiety disorders, obsessive-compulsive disorders, social phobias or panic states, sleep disorders, sexual dysfunction, psychoses, schizophrenia, migraine and other conditions associated with cephalic pain or other pain, raised intracranial pressure, epilepsy, personality disorders, Alzheimer disease, age-related behavioral disorders, behavioral disorders associated with dementia, organic mental disorders, mental disorders in childhood, aggressivity, age-related memory disorders, chronic fatigue syndrome, drug and alcohol addiction, obesity, bulimia, anorexia nervosa and premenstrual tension. In further embodiments, the disorder of the central nervous system is obesity. In further embodiments, the disorder of the central nervous system is Alzheimer disease. In further embodiments, the sexual dysfunction is Male erectile dysfunction.

In some embodiments, the damage to the central nervous system is by trauma, stroke, neurodegenerative diseases, toxic CNS diseases or infective CNS diseases. In further embodiments, the damage to the central nervous system is by encephalitis or meningitis.

In some embodiments, the cardiovascular disorder is thrombosis.

In some embodiments, the gastrointestinal disorder is dysfunction of gastrointestinal motility.

Another aspect of the present invention pertains to methods of producing a pharmaceutical composition comprising admixing at least one compound of the present invention and at least one pharmaceutically acceptable carrier.

Another aspect of the present invention pertains to compounds, as described herein, for use in a method of treatment of the human or animal body by therapy.

Another aspect of the present invention pertains to compounds, as described herein, for use in a method of treatment of disorders of the central nervous system; damage to the central nervous system; cardiovascular disorders; gastrointestinal disorders; diabetes insipidus or sleep apnea of the human or animal body by therapy.

Another aspect of the present invention pertains to use of compounds, as described herein, for the production of a medicament for use in the treatment of disorders of the central nervous system; damage to the central nervous system; cardiovascular disorders; gastrointestinal disorders; diabetes insipidus or sleep apnea.

Another aspect of the present invention pertains to the use of a compound of the present invention with agonist activity at the serotonin 5HT_{2C} receptor for the treatment of AD and AD related disorders. The compounds of the present invention can be used alone or in combination with another agent or agents (such as but not limited to AChE inhibitors) that are typically prescribed for AD.

### Combination Therapy—Prophylaxis and Treatment:

In the context of the present invention, a compound of Formula (Ia) or pharmaceutical composition thereof can be utilized to activate the 5HT_{2C} receptor that is associated with diseases, conditions and/or disorders as described herein. For example, activating the 5HT_{2C} receptor is useful in the treatment of obesity and/or overweight by decreasing food intake, inducing satiation (i.e., the feeling of fullness), controlling weight gain, decreasing body weight and/or affecting metabolism such that the recipient loses weight and/or maintains weight. Such compounds and pharmaceutical compositions can therefore be used in the context of disorders and/or diseases where weight gain is a component of a disease and/or disorder such as those listed herein. Furthermore, compounds and composition of the present invention can be used for the prophylaxis and/or treatment of Alzheimer Disease, erectile dysfunction and other 5HT_{2C} receptor associated diseases and/or disorders described herein. While the compounds of the invention can be administered as the sole active pharmaceutical agent (i.e., mono-therapy), they can also be used in combination with other pharmaceutical agents (i.e., combination-therapy) for the treatment of the diseases/conditions/disorders described herein. Therefore, another aspect of the present invention includes methods of prophylaxis and/or treatment comprising administering to an individual in need of prophylaxis and/or treatment a therapeutically effective amount of a compound of the present invention, for example Formula (Ia), in combination with one or more additional pharmaceutical agent as described herein.

Suitable pharmaceutical agents that can be used in combination with the compounds of the present invention include anti-obesity agents such as apolipoprotein-B secretion/microsomal triglyceride transfer protein (apo-B/MTP) inhibitors, MCR-4 agonists, cholescystokinin-A (CCK-A) agonists, serotonin and norepinephrine reuptake inhibitors (for example, sibutramine), sympathomimetic agensts, β₃ adrenergic receptor agonists, dopamine agonists (for example, bromocriptine), melanocyte-stimulating hormone receptor analogs, cannabinoid 1 receptor antagonists [for example, SR141716: N-(piperidin-1-yl)-5-(4-chlorophenyl)-1-(2,4-dichlorophenyl)-4-methyl-1H-pyrazole-3-carboxamide], melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion).

Other anti-obesity agents, including the agents set forth infra, are well known, or will be readily apparent in light of the instant disclosure, to one of ordinary skill in the art.

In some embodiments, the anti-obesity agents are selected from the group consisting of orlistat, sibutramine, bromocriptine, ephedrine, leptin, phentermine and pseudoephedrine. In a further embodiment, compounds of the present invention and combination therapies are administered in conjunction with exercise and/or a sensible diet.

It will be understood that the scope of combination-therapy of the compounds of the present invention with other anti-obesity agents, anorectic agents, appetite suppressant and related agents is not limited to those listed above, but includes in principle any combination with any pharmaceutical agent or pharmaceutical composition useful for the treatment of overweight and obese individuals.

Other suitable pharmaceutical agents, in addition to anti-obesity agents, that can be used in combination with the compounds of the present invention include agents useful in the treatment of concomitant diseases. For example, individuals that are over weight or obese increase their risk of morbidity and mortality arising from concomitant diseases, such as, but not limited to, congestive heart failure, type II diabetes, atherosclerosis, dyslipidemia, hyperinsulinemia, hypertension, insulin resistance, hyperglycemia, retinopathy, nephropathy and neuropathy. Treatment for one or more of the diseases cited herein include the use of one or more pharmaceutical agents known in the art belonging to the classes of drugs referred to, but not limited to, the following: sulfonylureas, meglitinides, biguanides, α-glucosidase inhibitors, peroxisome proliferators-activated receptor-γ (i.e., PPAR-γ) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin. In accordance to one aspect of the present invention, a compound of the present can be used in combination with a pharmaceutical agent or agents belonging to one or more of the classes of drugs cited herein.

It will be understood that the scope of combination-therapy of the compounds of the present invention with other pharmaceutical agents is not limited to those listed herein, supra or infra, but includes in principle any combination with any pharmaceutical agent or pharmaceutical composition useful for the treatment diseases, conditions or disorders that are linked to overweight and obese individuals.

Some embodiments of the present invention include methods of treatment of a disease, disorder or condition as described herein comprising administering to an individual in need of such treatment a therapeutically effect amount or dose of a compound of the present invention in combination with at least one pharmaceutical agent selected from the group consisting of: sulfonylureas, meglitinides, biguanides, α-glucosidase inhibitors, peroxisome proliferators-activated receptors (i.e., PPAR-γ) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin. In some embodiments, methods of the present invention include compounds of the present invention and the pharmaceutical agents are administered separately. In further embodiments, compounds of the present invention and the pharmaceutical agents are administered together.

Suitable pharmaceutical agents that can be used in conjunction with compounds of the present invention include α-glucosidase inhibitors. α-Glucosidase inhibitors belong to the class of drugs which competitively inhibit digestive enzymes such as α-amylase, maltase, α-dextrinase, sucrase, etc. in the pancreas and or small intestine. The reversible inhibition by α-glucosidase inhibitors retard, diminish or otherwise reduce blood glucose levels by delaying the digestion of starch and sugars. Some representative examples of α-glucosidase inhibitors include acarbose, N-(1,3-dihydroxy-2-propyl)valiolamine (generic name; voglibose), miglitol, and α-glucosidase inhibitors known in the art.

Suitable pharmaceutical agents that can be used in conjunction with compounds of the present invention include sulfonylureas. The sulfonylureas (SU) are drugs which promote secretion of insulin from pancreatic β cells by transmitting signals of insulin secretion via SU receptors in the cell membranes. Examples of the sulfonylureas include glyburide, glipizide, glimepiride and other sulfonylureas known in the art.

Suitable pharmaceutical agents that can be used in conjunction with compounds of the present invention include the meglitinides. The meglitinides are benzoic acid derivatives represent a novel class of insulin secretagogues. These agents target postprandial hyperglycemia and show comparable efficacy to sulfonylureas in reducing HbA_{1c}. Examples of meglitinides include repaglinide, nateglinide and other meglitinides known in the art.

Suitable pharmaceutical agents that can be used in conjunction with compounds of the present invention include the biguanides. The biguanides represent a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin, metformin, buformin, and biguanides known in the art.

Suitable pharmaceutical agents that can be used in conjunction with compounds of the present invention include the α-glucosidase inhibitors. The α-glucosidase inhibitors competitively inhibit digestive enzymes such as α-amylase, maltase, α-dextrinase, sucrase, etc. in the pancreas and or small intestine. The reversible inhibition by α-glucosidase inhibitors retard, diminish or otherwise reduce blood glucose levels by delaying the digestion of starch and sugars. Examples of α-glucosidase inhibitors include acarbose, N-(1,3-dihydroxy-2-propyl)valiolamine (generic name; voglibose), miglitol, and α-glucosidase inhibitors known in the art.

Suitable pharmaceutical agents that can be used in conjunction with compounds of the present invention include the peroxisome proliferators-activated receptor-γ (i.e., PPAR-γ) agonists. The peroxisome proliferators-activated receptor-γ agonists represent a class of compounds that activates the nuclear receptor PPAR-γ and therefore regulate the transcription of insulin-responsive genes involved in the control of glucose production, transport and utilization. Agents in the class also facilitate the regulation of fatty acid metabolism. Examples of PPAR-γ agonists include rosiglitazone, pioglitazone, tesaglitazar, netoglitazone, GW-409544, GW-501516 and PPAR-γ agonists known in the art.

Suitable pharmaceutical agents that can be used in conjunction with compounds of the present invention include the HMG-CoA reductase inhibitors. The HMG-CoA reductase inhibitors are agents also referred to as Statin compounds that belong to a class of drugs that lower blood cholesterol levels by inhibiting hydroxymethylglutalyl CoA (HMG-CoA) reductase. HMG-CoA reductase is the rate-limiting enzyme in cholesterol biosynthesis. The statins lower serum LDL concentrations by upregulating the activity of LDL receptors and are responsible for clearing LDL from the blood. Some representative examples the statin compounds include rosuvastatin, pravastatin and its sodium salt, simvastatin, lovastatin, atorvastatin, fluvastatin, cerivastatin, rosuvastatin, pitavastatin, BMS's “superstatin”, and HMG-CoA reductase inhibitors known in the art.

Suitable pharmaceutical agents that can be used in conjunction with compounds of the present invention include the angiotensin converting enzyme (ACE) inhibitors. The angiotensin converting enzyme inhibitors belong to the class of drugs that partially lower blood glucose levels as well as lowering blood pressure by inhibiting angiotensin converting enzymes. Examples of the angiotensin converting enzyme inhibitors include captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and angiotensin converting enzyme inhibitors known in the art.

Suitable pharmaceutical agents that can be used in conjunction with compounds of the present invention include the angiotensin II receptor antagonists. Angiotensin II receptor antagonists target the angiotensin II receptor subtype 1 (i.e., AT1) and demonstrate a beneficial effect on hypertension. Examples of angiotensin II receptor antagonists include losartan (and the potassium salt form), and angiotensin II receptor antagonists known in the art.

Other treatments for one or more of the diseases cited herein include the use of pharmaceutical agents known in the art belonging to the classes of drugs referred to, but not limited to, the following: amylin agonists (for example, pramlintide), insulin secretagogues (for example, GLP-1 agonists; exendin-4; insulinotropin (NN2211); dipeptyl peptidase inhibitors (for example, NVP-DPP-728), acyl CoA cholesterol acetyltransferase inhibitors (for example, Ezetimibe, eflucimibe, and like compounds), cholesterol absorption inhibitors (for example, ezetimibe, pamaqueside and like compounds), cholesterol ester transfer protein inhibitors (for example, CP-529414, JTT-705, CETi-1, and like compounds), microsomal triglyceride transfer protein inhibitors (for example, implitapide, and like compounds), cholesterol modulators (for example, NO-1886, and like compounds), bile acid modulators (for example, GT103-279 and like compounds) and squalene synthase inhibitors.

Squalene synthesis inhibitors belong to a class of drugs that lower blood cholesterol levels by inhibiting synthesis of squalene. Examples of the squalene synthesis inhibitors include (S)-α-[Bis[2,2-dimethyl-1-oxopropoxy)methoxy] phosphinyl]-3-phenoxybenzenebutanesulfonic acid, mono potassium salt (BMS-188494) and squalene synthesis inhibitors known in the art.

### Compositions of the Present Invention

According to a further aspect, the present invention also pertains to pharmaceutical compositions comprising one or more compounds of Formula (Ia) or any formulae disclosed herein, and one or more pharmaceutically acceptable carriers.

Some embodiments of the present invention include a method of producing a pharmaceutical composition comprising admixing at least one compound according to any of the compound embodiments disclosed herein and a pharmaceutically acceptable carrier.

Formulations may be prepared by any suitable method, typically by uniformly mixing the active compound(s) with liquids or finely divided solid carriers, or both, in the required proportions, and then, if necessary, forming the resulting mixture into a desired shape.

Conventional excipients, such as binding agents, fillers, acceptable wetting agents, tabletting lubricants, and disintegrants may be used in tablets and capsules for oral administration. Liquid preparations for oral administration may be in the form of solutions, emulsions, aqueous or oily suspensions, and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives, and flavorings and colorants may be added to the liquid preparations. Parenteral dosage forms may be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro, A. R., et al.).

While it is possible that a compound of the invention may, in an alternative use, be administered as a raw or pure chemical, it is preferable however to present the compound or active ingredient as a pharmaceutical formulation or composition further comprising a pharmaceutically acceptable carrier. Accordingly, another aspect of the present invention pertains to pharmaceutical compositions comprising a pharmaceutically acceptable carrier in combination with at least one compound according to Formula (Ia).

The invention further provides pharmaceutical formulations comprising a compound of the invention or a pharmaceutically acceptable salt or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof, and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a solvate or physiologically functional derivative thereof can be used as active ingredients in pharmaceutical compositions, specifically as 5HT_{2C} receptor agonists. By the term “active ingredient” is defined in the context of a “pharmaceutical composition” and shall mean a component of a pharmaceutical composition that provides the primary pharmacological effect, as opposed to an “inactive ingredient” which would generally be recognized as providing no pharmaceutical benefit.

The dose when using the compounds of the present invention can vary within wide limits, and as is customary and is known to the physician, it is to be tailored to the individual conditions in each individual case. It depends, for example, on the nature and severity of the illness to be treated, on the condition of the patient, on the compound employed or on whether an acute or chronic disease state is treated or on whether a further active compound is administered in addition to the compound of the present invention. Representative doses of the present invention include, but not limited to, about 0.001 mg to about 5000 mg, about 0.001 to about 2500 mg, about 0.001 to about 1000 mg, 0.001 to about 500 mg, 0.001 mg to about 250 mg, about 0.001 mg to 100 mg, about 0.001 mg to about 50 mg, and about 0.001 mg to about 25 mg. Multiple doses may be administered during the day, especially when relatively large amounts are deemed to be needed, for example 2, 3 or 4, doses. Depending on the individual and as deemed appropriate from the patient's physician or care-giver it may be necessary to deviate upward or downward from the doses described herein.

The amount of active ingredient, active salt or hydrate thereof, required for use in treatment will vary not only with the particular salt selected but also with the route of administration, the nature of the condition being treated and the age and condition of the patient and will ultimately be at the discretion of the attendant physician or clinician.

In general, one skilled in the art understands how to extrapolate in vivo data obtained in a model system, typically an animal model, to another, such as a human. Typically, animal models include, but are not limited to, rodent models. In some circumstances, these extrapolations may merely be based on the weight of the animal model in comparison to another, such as a mammal, preferably a human, however, more often, these extrapolations are not simply based on weights, but rather incorporate a variety of factors. Representative factors include, but are not limited to, the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds of the Formula (Ia) as part of combination-therapy. The dosage regimen for treating a disease condition with the compounds and/or compositions of the present invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the case of dropper or pipette, the formulation may be achieved by the patient whereby administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the Formula (Ia) or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the Formula (Ia) as an aerosol can be prepared by processes well-known to the person skilled in the art. For their preparation, for example, solutions or dispersions of the compounds of the Formula (Ia) in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed in Journal of Pharmaceutical Science, 66, 2 (1977); incorporated herein by reference in its entirety.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In general, the “pro-drug” approach is utilized to facilitate oral absorption. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Some embodiments of the present invention include a method of producing a pharmaceutical composition for “combination-therapy” comprising admixing at least one compound according to any of the compound embodiments disclosed herein, at least one pharmaceutical agent as described herein and a pharmaceutically acceptable carrier.

In some embodiments the pharmaceutical agents is selected from the group consisting of: apolipoprotein-B secretion/microsomal triglyceride transfer protein (apo-B/MTP) inhibitors, MCR-4 agonists, cholescystokinin-A (CCK-A) agonists, serotonin and norepinephrine reuptake inhibitors (for example, sibutramine), sympathomimetic agensts, 3 adrenergic receptor agonists, dopamine agonists (for example, bromocriptine), melanocyte-stimulating hormone receptor analogs, cannabinoid 1 receptor antagonists [for example, SR141716: N-(piperidin-1-yl)-5-(4-chlorophenyl)-1-(2,4-dichlorophenyl)-4-methyl-1H-pyrazole-3-carboxamide], melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion). In further embodiments, the pharmaceutical agent is selected from the group consisting of orlistat, sibutramine, bromocriptine, ephedrine, leptin, phentermine and pseudoephedrine.

In some embodiments the pharmaceutical agents is selected from the group consisting of: sulfonylureas, meglitinides, biguanides, α-glucosidase inhibitors, peroxisome proliferators-activated receptor-γ (i.e., PPAR-γ) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin.

It is noted that when the 5HT_{2C} receptor agonists are utilized as active ingredients in a pharmaceutical composition, these are not intended for use only in humans, but in other non-human mammals as well. Indeed, recent advances in the area of animal health-care indicate that consideration be given for the use of agents, such as 5HT_{2C} receptor agonists, for the treatment of obesity and related disorders in domestic animals (e.g., cats and dogs), and 5HT_{2C} receptor agonists in other domestic animals where no disease or disorder is evident (e.g., food-oriented animals such as cows, chickens, fish, etc.). Those of ordinary skill in the art are readily credited with understanding the utility of such compounds in such settings.

### Other Utilities

Another object of the present invention relates to radio-labeled compounds of Formula (Ia) that would be useful not only in radio-imaging but also in assays, both in vitro and in vivo, for localizing and quantitating the 5HT_{2C} receptor in tissue samples, including human, and for identifying 5HT_{2C} receptor ligands by inhibition binding of a radio-labeled compound. It is a further object of this invention to develop novel 5HT_{2C} receptor assays of which comprise such radio-labeled compounds.

The present invention embraces isotopically-labeled compounds of Formula (Ia) and any subgenera herein, such as but not limited to, Formula (Ia) through Formula (Is). An “isotopically” or “radio-labeled” compounds are those which are identical to compounds disclosed herein, but for the fact that one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro 5HT_{2C} receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled “radio-labeled compound” is a compound of Formula (Ia) that has incorporated at least one radionuclide; in some embodiments the radionuclide is selected from the group consisting of ³H, ¹⁴C, ¹²⁵I, ³⁵S and ⁸²Br.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Schemes supra and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the more scarce radio-isotope or nonradio-active isotope.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl (3H) products by treating appropriate precursors with high specific activity methyl iodide (3H). This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na ¹²⁵I. A represented procedure was reported by Zhu, D.-G. and co-workers in J. Org. Chem. 2002, 67, 943-948.

B. Ortho ¹²⁵Iodination of phenols—This procedure allows for the incorporation of ¹²⁵I at the ortho position of a phenol as reported by Collier, T. L. and co-workers in J. Labeled Compd Radiopharm. 1999, 42, S264-S266.

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. and co-workers in J. Labeled Compd Radiopharm. 2001, 44, S280-S282.

A radio-labeled 5HT_{2C} receptor compound of Formula (Ia) can be used in a screening assay to identify/evaluate compounds. In general terms, a newly synthesized or identified compound (i.e., test compound) can be evaluated for its ability to reduce binding of the “radio-labeled compound of Formula (Ia)” to the 5HT_{2C} receptor. Accordingly, the ability of a test compound to compete with the “radio-labeled compound of Formula (Ia)” for the binding to the 5HT_{2C} receptor directly correlates to its binding affinity.

The labeled compounds of the present invention bind to the 5HT_{2C} receptor. In one embodiment the labeled compound has an IC₅₀ less than about 500 μM, in another embodiment the labeled compound has an IC₅₀ less than about 100 μM, in yet another embodiment the labeled compound has an IC₅₀ less than about 10 μM, in yet another embodiment the labeled compound has an IC₅₀ less than about 1 μM, and in still yet another embodiment the labeled inhibitor has an IC₅₀ less than about 0.1 μM.

Other uses of the disclosed receptors and methods will become apparent to those in the art based upon, inter alia, a review of this disclosure.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### EXAMPLES

### Example 1

### Intracellular IP₃ Accumulation Assay

HEK293 cells were transfected in 15 cm sterile dishes with or without (control) 16 ug of human 5HT_{2C} receptor cDNA [for example see, Saltzman, A. G., et al. Biochem. Biophys. Res. Commun. 181, 1469-1478 (1991)] using 25 ul of lipofectamine. Cells were then incubated for 3-4 hours at 37° C./5% CO₂ and then transfection media was removed and replaced with 100 ul of DMEM. Cells were then plated onto 100 cm sterile dishes. The next day cells were plated into 96 well PDL microtiter plates at a density of 55K/0.2 mL. Six hours latter, media was exchanged with [³H]inositol (0.25 uCi/well) in inositol free DMEM and plates were incubated at 37° C./5% CO₂ overnight. The next day, wells were aspirated and 200 ul of DMEM containing test compound, 10 uM pargyline, and 10 mM LiCl was added to appropriate wells. Plates were then incubated at 37° C./5% CO₂ for three hours followed aspiration and by addition of fresh ice cold stop solution (1M KOH, 19 mM Na-borate, 3.8 mM EDTA) to each well. Plates were kept on ice for 5-10 min and the wells were neutralized by addition of 200 ul of fresh ice cold neutralization solution (7.5% HCl). Plates were then frozen until further processing is desired. The lysate was then transferred into 1.5 mL Eppendorf tubes and 1 mL of chloroform/methanol (1:2) was added/tube. The solution was vortexed for 15 seconds and the upper phase was applied to a Biorad AG1-X8™ anion exchange resin (100-200 mesh). First, the resin was washed with water at 1:1.25 W/V and 0.9 mL of upper phase was loaded onto the column. The column was then washed with 10 mL of 5 mM myo-inositol and 10 mL of 5 mM Na-borate/60 mM Na-formate. The inositol tris phosphates were eluted into scintillation vials containing 10 mL of scintillation cocktail with 2 mL of 0.1 M formic acid/1 M ammonium formate. The columns were regenerated by washing with 10 mL of 0.1 M formic acid/3M ammonium formate and rinsed twice with dd H₂O and stored at 4° C. in water.

The biological activities in the IP Accumulation Assay for several representative compounds are shown in Table 3 below:

TABLE 3

| | 5HT_{2C} (EC₅₀) |
|---|---|
| Compound No. | IP Accumulation Assay (nM) |
| 1 | 2 |
| 10 | 5 |

Certain compounds of the present invention have an EC₅₀ value in the IP accumulation assay in the range of less than about 200 nM.

Compounds of the present invention are selective for the 5HT_{2C} receptor compared to the 5HT_{2A} and 5HT_{2B} receptors; for example Compound 10 has an EC₅₀ value of about 1.7 μM against the 5HT_{2A} receptor and is essentially inactive against the 5HT_{2B} receptor.

### Example 2

### Inhibition of Basal Food Intake Rats

Male Sprague-Dawley rats (250-350 g) are deprived of food overnight prior to testing. Prior to food deprivation, the animals are weighed and separated into treatment groups in order to balance groups according to body weight. On the test day, animals are placed into individual cages (no bedding) at 9:00 am with free access to water. At 10:00 AM, animals are injected with test compound (p.o., i.p., or s.c.) and then presented with a pre-weighed amount of food in a dish either 60 min (p.o.) or 30 min (i.p. and s.c.) after drug administration. Food consumption over different time points is determined by weighing the food cup at 1, 2, 4, and 6 hr after the food is presented. Thus, food consumption is measured at 2, 3, 5, and 7 hr post-injection in p.o. studies, and at 1.5, 2.5, 4.5, and 6.5 hr post-injection in i.p. and s.c. studies.

### Example 3 Syntheses of Selected Compounds of the Invention

Illustrated syntheses for compounds of the present invention are shown in FIGS. 1 and 2 where the symbols have the same definitions as used throughout this disclosure.

The compounds of the invention and their synthesis are further illustrated by the following examples. The following examples are provided to further define the invention without, however, limiting the invention to the particulars of these examples. The compounds described herein, supra and infra, are named according to CS Chem Draw Ultra Version 7.0.1 or AutoNom 2000. In certain instances common names are used and it is understood that these common names would be recognized by those skilled in the art.

Chemistry: Proton nuclear magnetic resonance (¹H NMR) spectra were recorded on a Varian Mercury Vx-400 equipped with a 4 nucleus auto switchable probe and z-gradient or a Bruker Avance-400 equipped with a QNP (Quad Nucleus Probe) or a BBI (Broad Band Inverse) and z-gradient. Chemical shifts are given in parts per million (ppm) with the residual solvent signal used as reference. NMR abbreviations are used as follows: s=singlet, d=doublet, dd=doublet of doublet, dt=doublet of triplet, t=triplet, q=quartet, m=multiplet, br=broad. Microwave irradiations were carried out using the Emyrs Synthesizer (Personal Chemistry). Thin-layer chromatography (TLC) was performed on silica gel 60 F₂₅₄ (Merck), preparatory thin-layer chromatography (prep TLC) was preformed on PK6F silica gel 60 A 1 mm plates (Whatman), and column chromatography was carried out on a silica gel column using Kieselgel 60, 0.063-0.200 mm Merck). Evaporation was done in vacuo on a Buchi rotary evaporator. Celite 545® was used during palladium filtrations.

LCMS specs: 1) PC: HPLC-pumps: LC-10AD VP, Shimadzu Inc.; HPLC system controller: SCL-10A VP, Shimadzu Inc; UV-Detector: SPD-10A VP, Shimadzu Inc; Autosampler: CTC HTS, PAL, Leap Scientific; Mass spectrometer: API 150EX with Turbo Ion Spray source, AB/MDS Sciex; Software: Analyst 1.2. 2) Mac: HPLC-pumps: LC-8A VP, Shimadzu Inc; HPLC system controller: SCL-10A VP, Shimadzu Inc. UV-Detector: SPD-10A VP, Shimadzu Inc; Autosampler: 215 Liquid Handler, Gilson Inc; Mass spectrometer: API 150EX with Turbo Ion Spray source, AB/MDS Sciex Software: Masschrom 1.5.2.

### Example 3 Syntheses of Selected Compounds of the Invention

### Example 3.1

### Preparation of (R)-1-(3-Fluoro-5-thiophen-3-yl-phenyl)-2-methyl-piperazine

### Step A: Preparation of 3-(3-Bromo-5-fluoro-phenyl)-thiophene

A solution of 1,3-dibromo-5-fluorobenzene (0.50 g, 1.99 mmol), 3-thiopheneboronic acid (0.254 g, 1.99 mmol), 2.0 M aqueous Sodium carbonate (1.99 mL, 3.97 mmol) and Pd(PPh₃)₄ (115 mg, 0.10 mmol) in toluene (9.2 mL) and ethanol (2.4 mL), was heated at 100° C. for 1.5 hours by microwave. The product mixture was diluted with EtOAc (100 mL) filtered, washed with water (50 mL), brine (50 mL), dried with Na₂SO₄ and concentrated. Flash chromatography (2% EtOAc in hexane, silica) resulted in 400 mg of pure product as a clear oil.

### Step B: Preparation of (R)-1-(3-Fluoro-5-thiophen-3-yl-phenyl)-2-methyl-piperazine

A solution of 3-(3-bromo-5-fluoro-phenyl)-thiophene (200 mg, 0.78 mmol), (R)-3-methyl-piperazine-1-carboxylic acid tert-butyl ester (156 mg, 0.78 mmol), sodium tert-butoxide (112 mg, 1.17 mmol), 10% P(t-Bu)₃ in hexane (0.13 mL, 0.06 mmol), Pd₂(dba)₃ (36 mg, 0.04 mmol) in toluene (1.6 mL) was heated at 100° C. for 6 hours by microwave. The crude product mixture was filtered through a plug of silica, eluting with 5% EtOAc in hexane (20 mL) and then pure EtOAc (20 mL), and then the combined solutions were concentrated. The product was dissolved in CH₂Cl₂ (1.0 mL), treated with trifluoroacetic acid (0.5 mL) for 3 hours, and then passed through a column of NaHCO₃ and concentrated. The product was purified by HPLC and then triturated with 2 M HCl in ether to give 128 mg of a white solid. ¹H NMR (400 MHz, CD₃OD) δ 7.69 (s, 1H), 7.49-7.43 (m, 2H), 7.12 (s, 1H), 7.00 (d, J=10 Hz, 1H), 6.73 (d, J=11 Hz, 1H), 4.13 (m, 1H), 3.55-3.41 (m, 3H), 3.36-3.24 (m, 4H), 1.16 (d, J=7 Hz, 3H). MS calculated for C₁₅H₁₇FN₂S+H: 277, observed: 277.

### Example 3.2

### Preparation of (R)-1-(3-Fluoro-5-pyridin-3-yl-phenyl)-2-methyl-piperazine

By the same general procedure as example 3.1, (R)-1-(3-fluoro-5-pyridin-3-yl-phenyl)-2-methyl-piperazine was obtained from 3-pyridineboronic acid as a white solid. ¹H NMR (400 MHz, CD₃OD) δ 9.09 (s, 1H), 8.76 (d, J=5 Hz, 1H), 6.68 (d, J=8 Hz, 1H), 7.99 (dd, J=8, 5 Hz, 1H), 7.49-7.44 (m, 2H), 7.30 (dd, J=12, 9 Hz, 1H), 3.44 (bs, 8H). MS calculated for C₁₅H₁₆FN₃+H: 258, observed: 258.

### Example 3.3

### Preparation of (R)-1-(5-Fluoro-biphenyl-3-yl)-2-methyl-piperazine

By the same general procedure as example 3.1, (R)-1-(5-fluoro-biphenyl-3-yl)-2-methyl-piperazine was obtained from phenylboronic acid as a white solid. ¹H NMR (400 MHz, CD₃OD) δ 7.59 (d, J=7 Hz, 2H), 7.44 (dd, J=7, 7 Hz, 2H), 7.36 (dd, J=7, 7 Hz, 1H), 7.05 (s, 1H), 6.94 (d, J=10 Hz, 1H), 6.79 (d, J=11 Hz, 1H), 4.13 (m, 1H), 3.57-3.41 (m, 3H), 3.36-3.24 (m, 4H), 1.16 (d, J=7 Hz, 3H). MS calculated for C₁₇H₁₉FN₂+H: 271, observed: 271.

### Example 3.4

### Preparation of (R)-1-(5,2′-Difluoro-biphenyl-3-yl)-2-methyl-piperazine

By the same general procedure as example 3.1, (R)-1-(5,2′-Difluoro-biphenyl-3-yl)-2-methyl-piperazine was obtained from 2-fluorophenylboronic acid as a white solid. ¹H NMR (400 MHz, CD₃OD) δ 7.48 (dd, J=8, 8 Hz, 1H), 7.42-7.36 (m, 1H), 7.26 (dd, J=8, 8 Hz, 1H), 7.19 (dd, J=11, 8 Hz, 1H), 6.99 (s, 1H), 6.88-6.81 (m, 2H), 4.10 (m, 1H), 3.52-3.40 (m, 3H), 3.35-3.24 (m, 3H), 1.17 (d, J=7 Hz, 3H). MS calculated for C₁₇H₁₈F₂N₂+H: 289, observed: 289.

### Example 3.5

### Preparation of (R)-1-(3-Fluoro-5-furan-3-yl-phenyl)-2-methyl-piperazine

By the same general procedure as example 3.1, (R)-1-(3-Fluoro-5-furan-3-yl-phenyl)-2-methyl-piperazine was obtained from 3-furanboronic acid as a white solid. ¹H NMR (400 MHz, CD₃OD) δ 7.94 (s, 1H), 7.56 (s, 1H), 7.01 (s, 1H), 6.90 (d, J=10 Hz, 1H), 6.80 (s, 1H), 6.69 (d, J=11 Hz, 1H), 4.11 (m, 1H), 3.52-3.41 (m, 3H), 3.37-3.24 (m, 4H), 1.14 (d, J=7 Hz, 3H). MS calculated for C₁₅H₁₇FN₂O+H: 261, observed: 261.

### Example 3.6

### Preparation of (R)-1-(6-Fluoro-biphenyl-3-yl)-2-methyl-piperazine

By the same general procedure as example 3.1, R)-1-(6-Fluoro-biphenyl-3-yl)-2-methyl-piperazine was obtained from phenylboronic acid and 2-bromo-4-chloro-1-fluorobenzene as a white solid. ¹H NMR (400 MHz, CD₃OD) δ 7.52 (d, J=8 Hz, 2H), 7.44 (dd, J=8, 8 Hz, 2H), 7.37 (dd, J=7, 7 Hz, 1H), 7.20-7.11 (m, 3H), 3.66 (m, 1H), 3.44 (dd, J=12, 4 Hz, 1H), 3.36-3.26 (m, 4H), 3.09 (dd, J=14, 8 Hz, 1H), 1.04 (d, J=7 Hz, 3H). MS calculated for C₁₇H₁₉FN₂+H: 271, observed: 271.

### Example 3.7

### Preparation of (R)-1-(4-Fluoro-3-pyridin-3-yl-phenyl)-2-methyl-piperazine

By the same general procedure as example 3.1, (R)-1-(4-Fluoro-3-pyridin-3-yl-phenyl)-2-methyl-piperazine was obtained from 3-pyridineboronic acid and 2-bromo-4-chloro-1-fluorobenzene as a white solid. ¹H NMR (400 MHz, CD₃OD) δ 9.08 (s, 1H), 8.84 (d, J=6 Hz, 1 H), 8.73 (d, J=8 Hz, 1H), 8.09 (dd, J=8, 6 Hz, 1H), 7.38-7.28 (m, 3H), 3.88 (m, 1H), 3.46 (dd, J=13, 4 Hz, 1H), 3.42-3.33 (m, 5H), 3.17 (dd, J=13, 6 Hz, 1H), 1.09 (d, J=7 Hz, 3H). MS calculated for C₁₆H₁₈FN₃+H: 272, observed: 272.

### Example 3.8

### Preparation of (R)-2-Methyl-1-(5-phenyl-pyridin-3-yl)-piperazine

By the same general procedure as example 3.1, (R)-2-Methyl-1-(5-phenyl-pyridin-3-yl)-piperazine was obtained from phenylboronic acid and 3,5-dibromopyridine as a white solid. MS calculated for C₁₆H₁₉N₃+H: 254, observed: 254.

### Example 3.9

### Preparation of (R)-2-Methyl-1-(6-phenyl-pyridin-2-yl)-piperazine

By the same general procedure as example 3.1, (R)-2-Methyl-1-(6-phenyl-pyridin-2-yl)-piperazine was obtained from phenylboronic acid and 2,6-dibromopyridine as a white solid. ¹H NMR (400 MHz, CD₃OD) δ 7.93 (d, J=6 Hz, 2H), 7.61 (dd, J=8, 8 Hz, 1H), 7.45-7.37 (m, 3H), 7.17 (d, J=8 Hz, 1H), 6.60 (d, J=8 Hz, 1H), 4.34 (d, J=10 Hz, 1H), 3.60-3.10 (m, 6H), 1.39 (d, J=7 Hz, 3H). MS calculated for C₁₆H₁₉N₃+H: 254, observed: 254.

### Example 3.10

### Preparation of (R)-1-(2-Fluoro-biphenyl-3-yl)-2-methyl-piperazine

Step A: Preparation of (R)-1-(3-Chloro-2-fluoro-phenyl)-2-methyl-piperazine.

A solution of 2-Chloro-3-fluoroiodo-benzene (1.54 g, 6.0 mmol), (R)-3-methyl-piperazine-1-carboxylic acid tert-butyl ester (1.5 g, 7.5 mmol), sodium tert-butoxide (0.86 g, 9.0 mmol), 2-diphenylphosphino-2′-(N,N-dimethylamino)biphenyl (0.09 g, 0.24 mmol), Pd₂(dba)₃ (0.11 g, 0.12 mmol) in toluene (12 mL) was heated at 140° C. for 30 minutes by microwave. The crude product mixture was filtered through celite, eluting with EtOAc (100 mL), concentrated and then purified by column chromatography (5-30% EtOAc in hexanes, silica) to give 0.80 g of a white solid.

Step B: Preparation of (R)-1-(2-Fluoro-biphenyl-3-yl)-2-methyl-piperazine.

A solution of (R)-1-(3-Chloro-2-fluoro-phenyl)-2-methyl-piperazine (96 mg, 0.29 mmol), phenylboronic acid (37 mg, 0.35 mmol), K₃PO₄ (189 mg, 0.88 mmol), 2-dicyclohexylphosphino-2′,4′,6′-tri-1-propyl-1,1′-biphenyl (7 mg, 0.014 mmol) and Pd(OAc)₂ (1.6 mg, 0.007 mmol) in THF (1.0 mL) was heated at 150° C. for 30 minutes by microwave. The crude product mixture was filtered through celite, eluting with EtOAc (20 mL) and then concentrated. The crude product was dissolved in CH₂Cl₂ (1 mL) and treated with trifluoroacetic acid (1 mL) for 30 minutes, concentrated and then purified by HPLC to give 19 mg of a white solid. ¹H NMR (400 MHz, CD₃OD) δ 7.50 (d, J=8 Hz, 2H), 7.44 (dd, J=8, 8 Hz, 2H), 7.37 (dd, J=8, 8 Hz, 1H), 7.31-7.21 (m, 3H), 3.61 (m, 1H), 3.45-3.35 (m, 5H), 3.00 (dd, J=9, 12 Hz, 1H), 1.04 (d, J=6 Hz, 3H). MS calculated for C₁₇H₁₉FN₂+H: 271, observed: 271.

### Example 3.11

### Preparation of (R)-1-(5,3′-Difluoro-biphenyl-3-yl)-2-methyl-piperazine

By the same general procedure as example 3.1, (R)-1-(5,3′-Difluoro-biphenyl-3-yl)-2-methyl-piperazine was obtained from 3-fluorophenylboronic acid as a white solid. ¹H NMR (400 MHz, DMSO-d₆) δ 9.68 (bs, 1H), 9.16 (bs, 1H), 7.60-7.47 (m, 3H), 7.24 (dd, J=8, 8 Hz, 1H), 7.06 (s, 1H), 6.98 (d, J=10 Hz, 1H), 6.83 (d, J=12 Hz, 1H), 4.39 (m, 1H), 3.69 (d, J=13 Hz, 1 H), 3.31-3.18 (m, 4H), 3.03 (m, 1H), 1.16 (d, J=7 Hz, 3H). MS calculated for C₁₇H₁₈F₂N₂+H: 289, observed: 289.

### Example 3.12

### Preparation of (R)-1-(5,4′-Difluoro-biphenyl-3-yl)-2-methyl-piperazine

By the same general procedure as example 3.1, (R)-1-(5,4′-Difluoro-biphenyl-3-yl)-2-methyl-piperazine was obtained from 4-fluorophenylboronic acid as a white solid. ¹H NMR (400 MHz, DMSO-d₆) δ 9.75 (bs, 1H), 9.23 (bs, 1H), 7.74 (dd, J=6, 6 Hz, 2H), 7.29 (dd, J=9, 9 Hz, 2 H), 7.01 (s, 1H), 6.92 (d, J=10 Hz, 1H), 6.81 (d, J=12 Hz, 1H), 4.36 (m, 1H), 3.68 (d, J=13 Hz, 1H), 3.30-3.18 (m, 4H), 3.03 (m, 1H), 1.16 (d, J=7 Hz, 3H). MS calculated for C₁₇H₁₈F₂N₂+H: 289, observed: 289.

### Example 3.13

### Preparation of (R)-2-Methyl-1-(5,2′,6′-trifluoro-biphenyl-3-yl)-piperazine

By the same general procedure as example 3.1, (R)-2-Methyl-1-(5,2′,6′-trifluoro-biphenyl-3-yl)-piperazine was obtained from 2,6-difluorophenylboronic acid as a white solid. ¹H NMR (400 MHz, DMSO-d₆) δ 9.18 (bs, 1H), 8.72 (bs, 1H), 7.54-7.46 (m, 1H), 7.23 (dd, J=8, 8 Hz, 1H), 6.90 (d, J=10 Hz, 1H), 6.83 (s, 1H), 6.72 (d, J=7 Hz, 1H), 4.28 (m, 1H), 3.60 (d, J=13 Hz, 1H), 3.29-3.07 (m, 5H), 1.12 (d, J=7 Hz, 3H). MS calculated for C₁₇H₁₈F₃N₂+H: 307, observed: 307.

### Example 3.14

### Preparation of (R)-1-(4,3′-Difluoro-biphenyl-3-yl)-2-methyl-piperazine

By the same general procedure as example 3.1, (R)-1-(4,3′-Difluoro-biphenyl-3-yl)-2-methyl-piperazine was obtained from 3-fluorophenylboronic acid and 2-bromo-4-chloro-1-fluorobenzene as a white solid. ¹H NMR (400 MHz, DMSO-d₆) δ 9.27 (bs, 1H), 8.91 (bs, 1H), 7.52 (m, 1H), 7.41 (m, 2H), 7.26 (dd, J=8, 8 Hz, 2H), 7.12 (m, 1H), 7.07 (m, 1H), 4.04 (m, 1 H), 3.41 (m, 1H), 3.32 (m, 2H), 3.17 (m, 3H), 1.04 (d, J=7 Hz, 3H). MS calculated for C₁₇H₁₈F₂N₂+H: 289, observed: 289.

### Example 3.15

### Preparation of (R)-1-(4,4′-Difluoro-biphenyl-3-yl)-2-methyl-piperazine

By the same general procedure as example 3.1, (R)-1-(4,4′-Difluoro-biphenyl-3-yl)-2-methyl-piperazine was obtained from 4-fluorophenylboronic acid and 2-bromo-4-chloro-1-fluorobenzene as a white solid. ¹H NMR (400 MHz, DMSO-d₆) δ 9.24 (bs, 1H), 8.91 (bs, 1H), 7.62 (m, 2H), 7.31 (dd, J=10, 10 Hz, 2H), 7.21 (dd, J=10, 10 Hz, 1H), 7.09-7.02 (m, 2H), 4.01 (m, 1H), 3.39 (m, 1H), 3.30 (m, 2H), 3.17 (m, 3H), 1.03 (d, J=7 Hz, 3H). MS calculated for C₁₇H₁₈F₂N₂+H: 289, observed: 289.

### Example 3.16

### Preparation of (R)-1-Biphenyl-3-yl-2-methyl-piperazine

By the same general procedure as example 3.1, (R)-1-Biphenyl-3-yl-2-methyl-piperazine was obtained from phenylboronic acid and 3-bromo-chlorobenzene as a white solid. ¹H NMR (400 MHz, DMSO-d₆) δ 9.09 (bs, 1H), 8.70 (bs, 1H), 7.66 (d, J=8 Hz, 2H), 7.46 (dd, J=8, 8 Hz, 2H), 7.36 (dd, J=8, 8 Hz, 2H), 7.20 (s, 1H), 7.17 (d, J=8 Hz, 1H) 6.98 (d, J=8 Hz, 1H), 4.20 (m, 1 H), 3.53 (m, 1H), 3.32 (m, 2H), 3.22-3.12 (m, 3H), 1.06 (d, J=7 Hz, 3H). MS calculated for C₁₇H₂₀N₂+H: 253, observed: 253.

It is intended that each of the patents, applications, printed publications, and other published documents mentioned or referred to in this specification be herein incorporated by reference in their entirety.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

1. A compound of Formula (Ia): 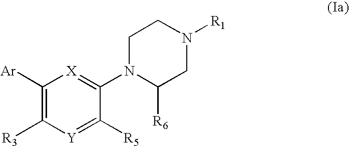
or a pharmaceutically acceptable salt, solvate, or hydrate thereof;
wherein:
X is N or CR₂;
Y is N or CR₄;
R₁ is H or C₁₋₄ alkyl;
R₂, R₃, R₄ and R₅ are each independently H, C₁₋₄ alkyl, C₁₋₄ haloalkyl or halogen provided that at least one group is other than H;
Ar is aryl or heteroaryl optionally substituted with 1, 2, 3, 4 or 5 substituents selected independently from the group consisting of C₁₋₄ acyl, C₁₋₄ acyloxy, C₁₋₄ acylthioxy, C₂₋₄ alkenyl, C₁₋₄ alkoxy, C₁₋₄ alkyl, C₁₋₄ alkylcarboxamido, C₁₋₄ alkylsulfinyl, C₁₋₄ alkylsulfonamide, C₁₋₄ alkylsulfonyl, C₁₋₄ alkylthio, amino, C₁₋₄ alkylamino, carbo-C₁₋₄-alkoxy, carboxamide, cyano, C₂₋₆ dialkylamino, C₁₋₄ haloalkoxy, C₁₋₄ haloalkyl, C₁₋₄ haloalkylsulfinyl, C₁₋₄ haloalkylsulfonyl, C₁₋₄ haloalkylthio, halogen, hydroxyl and thiol; and
R₆ is C₁₋₄ alkyl;
provided that when X and Y are both CH, R₁ and R₃ are both H, R₅ is F and R₆ is methyl, then Ar is a group other than phenyl.

2. The compound according to claim 1, or a pharmaceutically acceptable salt, solvate, or hydrate thereof, wherein X is N.

3. The compound according to claim 1, or a pharmaceutically acceptable salt, solvate, or hydrate thereof, wherein X is CR₂.

4. The compound according to claim 1, or a pharmaceutically acceptable salt, solvate, or hydrate thereof, wherein Y is N.

5. The compound according to claim 1, or a pharmaceutically acceptable salt, solvate, or hydrate thereof, wherein Y is CR₄.

6. The compound according to claim 1, or a pharmaceutically acceptable salt, solvate, or hydrate thereof, wherein R₁ is H.

7. The compound according to claim 1, or a pharmaceutically acceptable salt, solvate, or hydrate thereof, wherein R₁ is C₁₋₄ alkyl.

8. The compound according to claim 1, or a pharmaceutically acceptable salt, solvate, or hydrate thereof, wherein R₁ is methyl.

9. The compound according to claim 1, or a pharmaceutically acceptable salt, solvate, or hydrate thereof, wherein X is CR₂; Y is CR₄; R₂ is halogen; and R₃, R₄ and R₅ are each H.

10. The compound according to claim 1, or a pharmaceutically acceptable salt, solvate, or hydrate thereof, wherein X is CR₂; Y is CR₄; R₂ is F; and R₃, R₄ and R₅ are each H.

11. The compound according to claim 1, or a pharmaceutically acceptable salt, solvate, or hydrate thereof, wherein X is CR₂; Y is CR₄; R₃ is halogen; and R₂, R₄ and R₅ are each H.

12. The compound according to claim 1, or a pharmaceutically acceptable salt, solvate, or hydrate thereof, wherein X is CR₂; Y is CR₄; R₃ is F; and R₂, R₄ and R₅ are each H.

13. The compound according to claim 1, or a pharmaceutically acceptable salt, solvate, or hydrate thereof, wherein X is CR₂; Y is CR₄; R₄ is halogen; and R₂, R₃ and R₅ are each H.

14. The compound according to claim 1, or a pharmaceutically acceptable salt, solvate, or hydrate thereof, wherein X is CR₂; Y is CR₄; R₄ is F; and R₂, R₃ and R₅ are each H.

15. The compound according to claim 1, or a pharmaceutically acceptable salt, solvate, or hydrate thereof, wherein X is CR₂; Y is N; and R₂, R₃ and R₅ are each H.

16. The compound according to claim 1, or a pharmaceutically acceptable salt, solvate, or hydrate thereof, wherein X is N; Y is CR₄; and R₃, R₄ and R₅ are each H.

17. The compound according to claim 1, or a pharmaceutically acceptable salt, solvate, or hydrate thereof, wherein Ar is selected from the group consisting of thienyl, pyridinyl, phenyl and furanyl, each optionally substituted with 1 or 2 halo groups.

18. The compound according to claim 1, or a pharmaceutically acceptable salt, solvate, or hydrate thereof, wherein Ar is selected from the group consisting of thiophen-3-yl, pyridine-3-yl, phenyl, 2-fluorophenyl, furan-3-yl, 3-fluorophenyl, 4-fluorophenyl, and 2,6-difluorophenyl.

19. The compound according to claim 1, or a pharmaceutically acceptable salt, solvate, or hydrate thereof, wherein R₆ is methyl.

20. The compound according to claim 1, or a pharmaceutically acceptable salt, solvate, or hydrate thereof, wherein:
R₁ is H;
X is N or CR₂; and Y is N or CR₄; provided that not both X and Y are N;
R₂, R₃, R₄ and R₅ are each independently H or halogen provided that at least one group is halogen;
Ar is selected from the group consisting of thienyl, pyridinyl, phenyl and furanyl, each optionally substituted with 1 or 2 halo groups; and
R₆ is methyl.

21. The compound according to claim 1, or a pharmaceutically acceptable salt, solvate, or hydrate thereof, wherein:
R₁ is H;
X is N;
Y is CR₄;
R₃, R₄ and R₅ are each H;
Ar is selected from the group consisting of thiophen-3-yl, pyridine-3-yl, phenyl and furan-3-yl; and
R₆ is methyl.

22. The compound according to claim 1, or a pharmaceutically acceptable salt, solvate, or hydrate thereof, wherein:
R₁ is H;
X is CR₂;
Y is N;
R₂, R₃ and R₅ are each H;
Ar is selected from the group consisting of thiophen-3-yl, pyridine-3-yl, phenyl and furan-3-yl; and
R₆ is methyl.

23. The compound according to claim 1, or a pharmaceutically acceptable salt, solvate, or hydrate thereof, wherein:
R₁ is H;
X is CR₂;
Y is CR₄;
R₂, R₃, R₄ and R₅ are each independently H or halogen provided that at least one group is halogen;
Ar is selected from the group consisting of thiophen-3-yl, pyridine-3-yl, phenyl, 2-fluorophenyl, furan-3-yl, 3-fluorophenyl, 4-fluorophenyl, and 2,6-difluorophenyl; and
R₆ is methyl.

24. The compound of claim 1, selected from the group consisting of:
1-(3-Fluoro-5-thiophen-3-yl-phenyl)-2-methyl-piperazine;
1-(3-Fluoro-5-pyridin-3-yl-phenyl)-2-methyl-piperazine;
1-(3-Fluoro-5-furan-3-yl-phenyl)-2-methyl-piperazine;
1-(4-Fluoro-3-pyridin-3-yl-phenyl)-2-methyl-piperazine;
1-(5-Fluoro-biphenyl-3-yl)-2-methyl-piperazine;
1-(5,2′-Difluoro-biphenyl-3-yl)-2-methyl-piperazine;
1-(6-Fluoro-biphenyl-3-yl)-2-methyl-piperazine;
2-Methyl-1-(5-phenyl-pyridin-3-yl)-piperazine;
2-Methyl-1-(6-phenyl-pyridin-2-yl)-piperazine; and
1-(2-Fluoro-biphenyl-3-yl)-2-methyl-piperazine; or
a pharmaceutically acceptable salt, solvate, or hydrate thereof.

25. The compound of claim 1, selected from the group consisting of:
1-(5,3′-Difluoro-biphenyl-3-yl)-2-methyl-piperazine;
1-(5,4′-Difluoro-biphenyl-3-yl)-2-methyl-piperazine;
2-Methyl-1-(5,2′,6′-trifluoro-biphenyl-3-yl)-piperazine;
1-(4,3′-Difluoro-biphenyl-3-yl)-2-methyl-piperazine;
1-(4,4′-Difluoro-biphenyl-3-yl)-2-methyl-piperazine; and
1-Biphenyl-3-yl-2-methyl-piperazine; or
a pharmaceutically acceptable salt, solvate, or hydrate thereof.

26. A pharmaceutical composition comprising a compound according to any one of claims 1 to 25, or a pharmaceutically acceptable salt, solvate, or hydrate thereof, in combination with a pharmaceutically acceptable carrier.

27. A method of treating a 5HT_{2c} receptor associated disorder comprising administering to an individual in need of such treatment an effective amount of a compound according to any one of claims 1 to 25, or a pharmaceutically acceptable salt, solvate, or hydrate thereof.

28. The method according to claim 27, wherein said 5HT_{2C} receptor associated disorder is a central nervous system; damage to the central nervous system; cardiovascular disorder; gastrointestinal disorder; diabetes insipidus or sleep apnea.

29. The method according to claim 28, wherein said central nervous system is selected from the group consisting of depression, atypical depression, bipolar disorders, anxiety disorders, obsessive-compulsive disorders, social phobias or panic states, sleep disorders, sexual dysfunction, psychoses, schizophrenia, migraine and other conditions associated with cephalic pain or other pain, raised intracranial pressure, epilepsy, personality disorders, Alzheimer disease, age-related behavioral disorders, behavioral disorders associated with dementia, organic mental disorders, mental disorders in childhood, aggressivity, age-related memory disorders, chronic fatigue syndrome, drug and alcohol addiction, obesity, bulimia, anorexia nervosa and premenstrual tension.

30. The method according to claim 28, wherein said disorder of the central nervous system is obesity.

31. The method according to claim 28, wherein said disorder of the central nervous system is Alzheimer disease.

32. The method according to claim 28, wherein said disorder of the central nervous system is Male erectile dysfunction.

33. The method according to claim 27, wherein said individual is a human.

34. (canceled)

35. A method of decreasing food intake of an individual comprising administering to said individual a therapeutically effective amount of a compound according to any one of claims 1 to 25, or a pharmaceutically acceptable salt, solvate, or hydrate thereof.

36. A method of inducing satiety in an individual comprising administering to said individual a therapeutically effective amount of a compound according to any one of claims 1 to 25, or a pharmaceutically acceptable salt, solvate, or hydrate thereof.

37. A method of controlling weight gain of an individual comprising administering to said individual suffering from weight control a therapeutically effective amount of a compound according to any one of claims 1 to 25, or a pharmaceutically acceptable salt, solvate, or hydrate thereof.

38.-39. (canceled)

40. A method of producing a pharmaceutical composition comprising admixing at least one compound according to any one of claims 1 to 25, or a pharmaceutically acceptable salt, solvate, or hydrate thereof, and a pharmaceutically acceptable carrier.

41.-51. (canceled)

52. The method according to claim 30 wherein said individual is a human.

53. The method according to claim 35 wherein said individual is a human.

54. The method according to claim 36 wherein said individual is a human.

55. The method according to claim 37 wherein said individual is a human.
